SEQUENCE LISTING (26 sequences)

SEQ ID NO: 1 - protein, 278 aa, Artificial Sequence
  aldose reductase [Pyrococcus furiosus DSM 3638]
MKRVNAFNDLKRIGDDKVTAIGMGTWGIGGRETPDYSRDKESIEAIRYGLELGMNLIDTAEFYGAGHAEEIVGEAIKEFEREDIFIVSKVWPTHFGYEEAKKAARASAKRLGTYIDLYLLHWPVDDFKKIEETLHALEDLVDEGVIRYIGVSNFNLELLQRSQEVMRKYEIVANQVKYSVKDRWPETTGLLDYMKREGIALMAYTPLEKGTLARNECLAKIGEKYGKTAAQVALNYLIWEENVVAIPKASNKEHLKENFGAMGWRLSEEDREMARRCV

SEQ ID NO: 2 - protein, 353 aa, Artificial Sequence
  Pichia methanolica
MNWEKVPQELYTRLGSSGLQISKIIVGCMSFGTKAWGGDWVLEDEDEIFAIMKKAYDQGIRTFDTADSYSNGVSERLLGKFIRKYNIDRSKLVILTKVFFPAPEEYESFSFFNHNFPGHELVNRSGLSRKHILDSAAASVERLGTYIDVLQIHRYDPNTPAEETMEALNDCIKQGLTRYIGASTMRAYQFIKYQNVAEKHGWAKFISMQSYYSLLYREEEAELIAYCNETGVGLIPWSPNAGGFLTRPVSKQDTARSASGAAALYGLEPFSEADKAIIDRVEELSKKKGVSMASVALAWVISKNSWPIIGFSKPGRVDDALDGFKLKLTEEDIKFLEEPYVPKPLPRLYSVIL

SEQ ID NO: 3 - protein, 267 aa, Artificial Sequence
  oxidoreductase [Saccharomyces cerevisiae S288c]
MSQGRKAAERLAKKTVLITGASAGIGKATALEYLEASNGDMKLILAARRLEKLEELKKTIDQEFPNAKVHVAQLDITQAEKIKPFIENLPQEFKDIDILVNNAGKALGSDRVGQIATEDIQDVFDTNVTALINITQAVLPIFQAKNSGDIVNLGSIAGRDAYPTGSIYCASKFAVGAFTDSLRKELINTKIRVILIAPGLVETEFSLVRYRGNEEQAKNVYKDTTPLMADDVADLIVYATSRKQNTVIADTLIFPTNQASPHHIFRG

SEQ ID NO: 4 - protein, 246 aa, Artificial Sequence
  acetoacetyl-CoA reductase [Cupriavidus necator]
MTQRIAYVTGGMGGIGTAICQRLAKDGFRVVAGCGPNSPRREKWLEQQKALGFDFIASEGNVADWDSTKTAFDKVKSEVGEVDVLINNAGITRDVVFRKMTRADWDAVIDTNLTSLFNVTKQVIDGMADRGWGRIVNISSVNGQKGQFGQTNYSTAKAGLHGFTMALAQEVATKGVTVNTVSPGYIATDMVKAIRQDVLDKIVATIPVKRLGLPEEIASICAWLSSEESGFSTGADFSLNGGLHMG

SEQ ID NO: 5 - protein, 272 aa, Artificial Sequence
  NADPH-dependent 3-quinuclidinone reductase [Rhodotorula mucilaginosa]
MSSPSDGPFPKATPQLPNSVFDMFSMKGKVTAITGGGGGIGFAAAEAIAEAGGDVALLYRSAPNMEERSAELAKRFGVKVKSYQCEVTEHESVKQAIEAVEKDFGRLDCYIANAGGGVPGSINPDYPLEAWHKTQSVNLHSTFYAARECARIFKAQGSGSFIATTSISARIVNVPYDQPAYNSSKAAVVHFCRSLARDWRNFARVNTISPGFFDTPMGPSDKAVEDVLYQKSVLGRAGDVKELKAAYLYLASNASTYTTGADLLIDGGYCLT

SEQ ID NO: 6 - protein, 256 aa, Artificial Sequence
  hypothetical protein YJR096W [Saccharomyces cerevisiae S288c]
MVPKFYKLSNGFKIPSIALGTYDIPRSQTAEIVYEGVKCGYRHFDTAVLYGNEKEVGDGIIKWLNEDPGNHKREEIFYTTKLWNSQNGYKRAKAAIRQCLNEVSGLQYIDLLLIHSPLEGAVDEGLVKSIGVSNYGKKHIDELLNWPELKHKPVVNQIEISPWIMRQELADYCKSKGLVVEAFAPLCHGYKMTNPDLLKVCKEVDRNPGQVLIRWSLQHGYLPLPKTKTVKRLEGNLAAYNFELSDEQMKFLDHAP

SEQ ID NO: 7 - protein, 261 aa, Artificial Sequence
  glucose dehydrogenase [Bacillus megaterium]
MYTDLKDKVVVVTGGSKGLGRAMAVRFGQEQSKVVVNYRSNEEEALEVKKEIEQAGGQAIIVRGDVTKEEDVVNLVETAVKEFGTLDVMINNAGVENPVPSHELSLENWNQVIDTNLTGAFLGSREAIKYFVENDIKGNVINMSSVHEMIPWPLFVHYAASKGGMKLMTETLALEYAPKGIRVNNIGPGAIDTPINAEKFADPEQRADVESMIPMGYIGNPEEIASVAAFLASSQASYVTGITLFADGGMTKYPSFQAGRG

SEQ ID NO: 8 - protein, 363 aa, Artificial Sequence
  Aad14p [Saccharomyces cerevisiae S288c]
MTDLFKPLPEPPTELGRLRVLSKTAGIRVSPLILGGASIGDAWSGFMGSMNKEQAFELLDAFYEAGGNCIDTANSYQNEESEIWIGEWMASRKLRDQIVIATKFTGDYKKYEVGGGKSANYCGNHKRSLHVSVRDSLRKLQTDWIDILYIHWWDYMSSIEEVMDSLHILVQQGKVLYLGVSDTPAWVVSAANYYATSHGKTPFSVYQGKWNVLNRDFERDIIPMARHFGMALAPWDVMGGGRFQSKKAMEERKKNGEGLRTFVGGPEKIAEEHGTESVTAIAIAYVRSKAKNVFPLIGGRKIEHLKQNIEALSIKLTPEQIEYLESIVPFDVGFPKSLIGDDPAVTKKLSPLTSMSARIAFDN

SEQ ID NO: 9 - protein, 366 aa, Artificial Sequence
  putative hedroxyacid dehydrogenase [Saccharomyces cerevisiae S288c]
MCDSPATTGKPTILFIADPCETSATLNSKAFKEKFRILRYQLDTKEAFLNFLERHEQDKICAIYAGFPAFKKIGGMTRSIIEHKSFPRKNLKCIVLCSRGYDGWDLDTLRKHEIRLYNYQDDENEKLIDDLKLHQVGNDVADCALWHILEGFRKFSYYQKLSRETGNTLTARAKAAEKSGFAFGHELGNMFAESPRGKKCLILGLGSIGKQVAYKLQYGLGMEIHYCKRSEDCTMSQNESWKFHLLDETIYAKLYQFHAIVVTLPGTHCNPGLILVNLGRGKILDLRAVSDALVTGRINHLGLDVFNKEPEIDEKIRSSDRLTSITPHLGSATKDVFEQSCELALTRILRVVSGEAASDEHFSRVV

SEQ ID NO: 10 - protein, 314 aa, Artificial Sequence
  trifunctional aldehyde reductase/xylose reductase/glucose 1-dehydrogenase (NADP(+)) [Saccharomyces cerevisiae S288c]
MSSLVTLNNGLKMPLVGLGCWKIDKKVCANQIYEAIKLGYRLFDGACDYGNEKEVGEGIRKAISEGLVSRKDIFVVSKLWNNFHHPDHVKLALKKTLSDMGLDYLDLYYIHFPIAFKYVPFEEKYPPGFYTGADDEKKGHITEAHVPIIDTYRALEECVDEGLIKSIGVSNFQGSLIQDLLRGCRIKPVALQIEHHPYLTQEHLVEFCKLHDIQVVAYSSFGPQSFIEMDLQLAKTTPTLFENDVIKKVSQNHPGSTTSQVLLRWATERLLGNLEIEKKFTLTEQELKDISALNANIRFNDPWTWLDGKFPTFA

SEQ ID NO: 11 - protein, 275 aa, Artificial Sequence
  2,5-diketo-D-gluconate reductase A [Escherichia coli str. K-12 substr. DH10B]
MANPTVIKLQDGNVMPQLGLGVWQASNEEVITAIQKALEVGYRSIDTAAAYKNEEGVGKALKNASVNREELFITTKLWNDDHKRPREALLDSLKKLQLDYIDLYLMHWPVPAIDHYVEAWKGMIELQKEGLIKSIGVCNFQIHHLQRLIDETGVTPVINQIELHPLMQQRQLHAWNATHKIQTESWSPLAQGGKGVFDQKVIRDLADKYGKTPAQIVIRWHLDSGLVVIPKSVTPSRIAENFDVWDFRLDKDELGEIAKLDQGKRLGPDPDQFGG

SEQ ID NO: 12 - protein, 267 aa, Artificial Sequence
  2,5-diketo-D-gluconate reductase B [Escherichia coli str. K-12 substr. DH10B]
MAIPAFGLGTFRLKDDVVISSVITALELGYRAIDTAQIYDNEAAVGQAIAESGVPRHELYITTKIWIENLSKDKLIPSLKESLQKLRTDYVDLTLIHWPSPNDEVSVEEFMQALLEAKKQGLTREIGISNFTIPLMEKAIAAVGAENIATNQIELSPYLQNRKVVAWAKQHGIHITSYMTLAYGKALKDEVIARIAAKHNATPAQVILAWAMGEGYSVIPSSTKRKNLESNLKAQNLQLDAEDKKAIAALDCNDRLVSPEGLAPEWD

SEQ ID NO: 13 - protein, 299 aa, Artificial Sequence
  Gcy1p [Saccharomyces cerevisiae S288c]
MPATLHDSTKILSLNTGAQIPQIGLGTWQSKENDAYKAVLTALKDGYRHIDTAAIYRNEDQVGQAIKDSGVPREEIFVTTKLWCTQHHEPEVALDQSLKRLGLDYVDLYLMHWPARLDPAYIKNEDILSVPTKKDGSRAVDITNWNFIKTWELMQELPKTGKTKAVGVSNFSINNLKDLLASQGNKLTPAANQVEIHPLLPQDELINFCKSKGIVVEAYSPLGSTDAPLLKEPVILEIAKKNNVQPGHVVISWHVQRGYVVLPKSVNSTEDFEAINNISKEKGEKRVVHPNWSPFEVFK

SEQ ID NO: 14 - DNA, 805 bp, Artificial Sequence
  Pyrococcus furiosus DSM 3638
atgaggccagttaattaagaggtaccatatgaaacgcgtgaatgcctttaatgatctgaaacgcattggtgatgataaagttaccgcaattggtatgggcacctggggtattggtggtcgtgaaacaccggattatagccgtgataaagaaagcattgaagccattcgtattggtggtcgtgaaacaccggattatagccgtgataaagaaagcattgaagccattcgttatggtctggaactgggcatgaatctgattgataccgcagaattttatggtgcaggccatgcagaagaaattgttggcgaagccatcaaagaatttgaacgcgaggatatctttattgttagcaaagtgtggccgacccattttggttatgaagaagccaaaaaagcagcacgtgcaagttatattggcgtgagcaactttaatctggaactgctgcagcgtagccaagaagttatgcgcaaatacgaaattgttgccaaccaggtgaaatatagcgttaaagatcgttggcctgaaaccaccggtctgctggattatatgaaacgtgaaggtattgcactgatggcatatacaccgctggaaaaaggcaccctggcacgtaatgaatgtctggccaaaattggcgaaaaatatggtaaaaccgcagcacaggttgcactgaattatctgatctgggaagaaaatgttgttgcaattccgaaagccagcaacaaagaacatctgaaagaaaattttggtgcaatgggttggcgtctgagcgaagaggatcgtgaaatggcacgtcgttgtgtttaa

SEQ ID NO: 15 - DNA, 1090 bp, Artificial Sequence
  Pichia methanolica
atgaggccagttaattaagaggtaccatatgaattgggaaaaagtgccgcaggaactgtatacccgtctgggtagcagcggtctgcagattagcaaaattattgtgggttgtatgagctttggcaccaaagcatggggtggtgattgggttctggaagatgaagatgaaatttttgccattatgaaaaaagcctatgatcagggtattcgtacctttgataccgcagatagctatagcaatggtgttagcgaacgtctgctgggtaaattcatccgcaaatacaacattgatcgcagcaaactggttattctgaccaaagttttttttccggcaccggaagaatatgaaagcttcagcttttttaaccataactttccgggtcatgaactggttaatcgtagcggtctgagccgtaaacatattctggatagcgcagcagcaagcgttgaacgtctgggcacctatattgatgttctgcagatccatcgttatgatccgaatacaccggctgaagaaacaatggaagccctgaacgattgtattaaacagggtctgacccgttatattggtgcaagcaccatgcgtgcctatcagttcattaaatatcagaacgtggccgaaaaacatggttgggccaaatttattagcatgcagagctattatagcctgctgtatcgtgaagaagaagcagaactgattgcctattgcaatgaaaccggtgttggtctgattccgtggagcccgaatgccggtggttttctgacccgtccggttagcaaacaggataccgcacgtagcgcaagcggtgcagcagcactgtatggtctggaaccgtttagcgaagcagataaagccattattgatcgtgtggaagaactgagcaaaaaaaaaggtgttagcatggcaagcgttgcactggcatgggttattagcaaaaacagctggccgattattggttttagcaaaccgggtcgtgttgatgatgcactggatggctttaaactgaaactgaccgaagaggatatcaaattcctggaagaaccgtatgttccgaaaccgctgcctcgtctgtatagcgttattctgtaa

SEQ ID NO: 16 - DNA, 832 bp, Artificial Sequence
  Saccharomyces cerevisiae S288c
atgaggccagttaattaagaggtaccatatgagccagggtcgtaaagcagcagaacgtctggcaaaaaaaaccgttctgattaccggtgcaagcgcaggtattggtaaagcaaccgcactggaatatctggaagcaagcaatggcgatatgaaactgattctggcagcacgtcgtctggaaaaactggaagaactgaaaaaaaccatcgatcaggaatttccgaacgcaaaagttcatgttgcacagctggatattacccaggcagaaaaaatcaaaccgtttatcgaaaatctgccgcaggaattcaaagatatcgatattctggtgaataatgcaggtaaagcactgggtagcgatcgtgttggtcagattgcaaccgaagatatccaggatgtgtttgataccaatgtgaccgcactgattaatattacacaggccgttctgccgatttttcaggcaaaaaacagcggtgatattgtgaatctgggtagcattgcaggtcgtgatgcatatccgaccggtagcatttattgtgcaagcaaatttgcagttggtgcatttaccgacagtctgcgcaaagaactgattaataccaaaatccgcgttattctgattgcaccgggtctggttgaaaccgaattcagcctggttcgttatcgtggtaatgaagaacaggccaaaaacgtgtataaagataccacaccgctgatggcagatgatgttgccgatctgattgtttatgcaaccagccgtaaacagaataccgttattgccgataccctgatttttccgaccaatcaggcatctccgcatcatatttttcgtggttaa

SEQ ID NO: 17 - DNA, 769 bp, Artificial Sequence
  Cupriavidus necator
atgaggccagttaattaagaggtaccatatgacccagcgtattgcctatgttaccggtggtatgggtggtattggcaccgcaatttgtcagcgtctggcaaaagatggttttcgtgttgttgcaggttgtggtccgaattctccgcgtcgtgaaaaatggctggaacagcagaaagcactgggttttgattttattgccagcgaaggtaatgttgcagattgggatagcaccaaaaccgcctttgataaagttaaaagcgaagtgggtgaagttgatgtgctgattaacaatgcaggtattacccgtgatgttgtgtttcgcaaaatgacccgtgccgattgggatgcagttattgataccaatctgaccagcctgtttaatgttaccaaacaggtgattgatggtatggcagatcgtggttggggtcgtattgttaatattagcagcgtgaatggtcagaaaggtcagtttggtcagaccaattatagcaccgcaaaagcaggtctgcatggttttacaatggcactggcacaggaagttgcaaccaaaggcgttaccgttaataccgtttctccgggttatattgccaccgatatggttaaagcaattcgtcaggatgtgctggataaaattgttgccaccattccggttaaacgtctgggtctgccggaagaaattgcaagcatttgtgcatggctgagcagcgaagaaagcggttttagcacaggtgcagattttagcctgaatggtggtctgcacatgggttaa

SEQ ID NO: 18 - DNA, 847 bp, Artificial Sequence
  Rhodotorula mucilaginosa
atgaggccagttaattaagaggtaccatatgagcagcccgtctgatggtccgtttccgaaagcaacaccgcagctgccgaatagcgtttttgacatgtttagcatgaaaggtaaagttaccgcaattaccggtggtggtggtggcattggttttgcagcagcagaagcaattgccgaagccggtggtgatgttgcactgctgtatcgtagcgcaccgaatatggaagaacgtagcgcagaactggcaaaacgttttggtgtgaaagtgaaaagctatcagtgcgaagttaccgaacatgaaagcgttaaacaggcaattgaagccgtggaaaaagattttggtcgcctggattgttatattgcaaatgcgggtggtggtgttccgggtagcattaatccggattatccgctggaagcatggcataaaacccagagcgttaatctgcatagcaccttttatgcagcacgtgaatgcgcacgtatttttaaagcacagggcagcggtagctttattgcaaccacctctattagcgcacgtattgtgaatgttccgtatgatcagcctgcatataatagcagcaaagcagccgttgttcatttttgtcgtagcctggcacgtgattggcgtaattttgcccgtgttaataccattagccctggtttttttgataccccgatgggtccgagcgataaagcagttgaagatgtgctgtatcagaaaagcgttctgggtcgtgccggtgatgttaaagaactgaaagcagcatatctgtatctggcaagcaatgcaagcacctataccaccggtgcagatctgctgattgatggtggttattgtctgacctaa

SEQ ID NO: 19 - DNA, 849 bp, Artificial Sequence
  Saccharomyces cerevisiae S288c
atggttcctaagttttacaaactttcaaacggcttcaaaatcccaagcattgctttgggaacctacgatattccaagatcgcaaacagccgaaattgtgtatgaaggtgtcaagtgcggctaccgtcatttcgatactgctgttctttatggtaatgagaaggaagttggcgatggtatcattaaatggttgaacgaagatccagggaaccataaacgtgaggaaatcttctacactactaaattatggaattcgcaaaacggatataaaagagctaaagctgccattcggcaatgtttgaatgaagtctcgggcttgcaatacatcgatcttcttttgattcattcgccactggaaggttctaaattaaggttggaaacttggcgcgccatgcaagaagcggttgatgaaggattggttaagtctataggggtttccaactatgggaaaaagcacattgatgaacttttgaactggccagaactgaagcacaagccagtggtcaaccaaatcgagatatcaccttggattatgagacaagaattagcagattactgtaaatctaaaggtctcgtcgtcgaagcctttgccccattgtgtcacggctacaaaatgactaatccagatttattaaaagtttgcaaagaggtggaccgtaatccaggtcaagttttgattcgttggtctttacaacacggttatttaccactaccgaagactaaaactgtgaagaggttagaaggtaaccttgcagcctacaactttgaactgtcagacgaacagatgaaatttcttgatcatcctgatgcttatgagcctaccgattgggaatgcacagacgcgccataa

SEQ ID NO: 20 - DNA, 789 bp, Artificial Sequence
  Bacillus megaterium
atgtataccgacctgaaagataaagttgttgttgtgaccggtggtagcaaaggtctgggtcgtgcaatggcagttcgttttggtcaggaacagagcaaagttgttgtgaattatcgcagcaatgaagaagaagccctggttggtcaggaacagagcaaagttgttgtgaattatcgcagcaatgaagaagaagccctggccaaagaagaggacgttgttaatctggttgaaaccgcagttaaagaatttggcaccctggatgtgatgattaataatgccggtgttgaaaatccggttccgagccatgaactgagcctggaaaattggaatcaggtgattgataccaatctgaccggtgcatttctgggtagccgtgaagccattaaatattttgtggaaaatgatattaaaggcaatgtgatcaatatgagcagcgttcatgaaatgattccgtggcctctgtttgttcattatgcagcaagcaaaggtggtatgaaactgatgaccgaaaccctggcactggaatatgcaccgaaaggtattcgtgtgaataatattggtccgggtgcaattgataccccgatcaatgcagaaaaatttgcagatccggaacagcgtgcagatgttgaaagcatgattccgatgggttatattggcaatccggaagaaattgcaagcgttgcagcatttctggcaagcagccaggcaagctatgttaccggtattaccctgtttgcagatggtggtatgaccaaatatccgagctttcaggcaggtcgtggttaataa

SEQ ID NO: 21 - DNA, 1131 bp, Artificial Sequence
  Saccharomyces cerevisiae S288c
atgactgacttgtttaaacctctacctgaaccacctaccgaattgggacgtctcagggttctttctaaaactgccggcataagggtttcaccgctaattctgggaggagcttcaatcggcgacgcatggtcaggctttatgggctctatgaataaggaacaggcctttgaacttcttgatgctttttatgaagctggaggtaattgtattgatactgcaaacagttaccaaaatgaagagtcagagatttggataggtgaatggatggcatcaagaaaactgcgtgaccagattgtaattgccaccaagtttaccggagattataagaagtatgaagtaggtggtggtaaaagtgccaactactgtggtaatcacaagcgtagtttacatgtgagtgtgagggattctctccgcaaattgcaaactgattggattgatatactttacattcactggtgggattatatgagttcaatcgaagaagttatggatagtttgcatattttagttcagcagggcaaggtcctatatttaggagtatctgatacacctgcttgggttgtttctgcggcaaattactacgctacatctcatggtaaaactccttttagcgtctatcaaggtaaatggaatgtattgaacagggactttgagcgtgatattattccaatggctaggcattttggtatggctctagccccatgggatgtcatgggaggtggaagatttcagagtaaaaaagcaatggaagaacggaagaagaatggagagggtctgcgtacttttgtgggtggccccgaacaaacagaattggaggttaaaatcagcgaagcattgactaaaattgctgaggaacatggaacagagtctgttactgctatcgctattgcctatgttcgctctaaagcgaaaaatgttttcccattgattggaggaaggaaaattgaacatctcaagcagaacattgaggctttgagtattaaattaacaccggaacaaatagaatacctggaaagtattgttccttttgatgttggctttcccaaaagtttaataggagatgacccagcggtaaccaagaagctttcacccctcacatcgatgtctgccaggatagcttttgacaattag

SEQ ID NO: 22 - DNA, 1140 bp, Artificial Sequence
  Saccharomyces cerevisiae S288c
atgtgcgattctcctgcaacgactggaaagcctactattcttttcatcgcagatccgtgcgaaacatcagccacacttaattccaaggcattcaaagagaagttcaggatcttgcgctatcagctggacaccaaagaagcatttcttaactttttagaaaggcatgaacaagacaaaatatgtgccatttatgctgggtttccggcattcaaaaaaatcggtgggatgactcgaagtatcatcgaacacaagtcatttccaaggaaaaatttaaaatgtatcgtgctttgctcaagaggttacgacggatgggatctggatacattacgcaagcatgaaattcgattatacaactaccaagacgatgaaaatgaaaaattgatagacgatttaaagcttcatcaagtcggtaatgatgtggcagattgtgccttgtggcacattctggagggctttagaaagttctcctattaccaaaaacttagtagagaaactggaaatacattaactgcaagggcgaaagctgcagaaaagagcggatttgcttttggccatgaactggggaatatgtttgctgaatcaccaagaggaaagaaatgcttaattcttggtttaggaagtattggaaagcaagtagcctacaagttgcaatacgggctaggaatggaaatacattattgcaaaagaagcgaagattgcacaatgagtcaaaacgaaagctggaaatttcatttgctagatgaaacaatatatgcaaaactataccagtttcatgcaatcgtggtcacattgccgggaactccacaaacagaacatttaatcaacaggaaatttttggaacactgcaatccaggcctaattttagtcaacttgggaagaggtaaaattttggacttgcgggctgtttctgacgccttggtaacgggacgaatcaaccatctcggtttagacgtctttaataaagaaccagaaatagatgaaaaaatcagatcttctgatagacttacttcaattactccgcatttgggtagtgcgacaaaggatgtttttgagcaaagttgtgaactggcattgacaagaatcttacgggtagtgtctggggaagccgcaagcgatgagcatttctcccgtgtagtttga

SEQ ID NO: 23 - DNA, 984 bp, Artificial Sequence
  Saccharomyces cerevisiae S288c
atgtcttcactggttactcttaataacggtctgaaaatgcccctagtcggcttagggtgctggaaaattgacaaaaaagtctgtgcgaatcaaatttatgaagctatcaaattaggctaccgtttattcgatggtgcttgcgactacggcaacgaaaaggaagttggtgaaggtatcaggaaagccatctccgaaggtcttgtttctagaaaggatatatttgttgtttcaaagttatggaacaattttcaccatcctgatcatgtaaaattagctttaaagaagaccttaagcgatatgggacttgattatttagacctgtattatattcacttcccaatcgccttcaaatatgttccatttgaagagaaataccctccaggattctatacgggcgcagatgacgagaagaaaggtcacatcaccgaagcacatgtaccaatcatagatacgtaccgggctctggaagaatgtgttgatgaaggcttgattaagtctattggtgtttccaactttcagggaagcttgattcaagatttattacgtggttgtagaatcaagcccgtggctttgcaaattgaacaccatccttatttgactcaagaacacctagttgagttttgtaaattacacgatatccaagtagttgcttactcctccttcggtcctcaatcattcattgagatggacttacagttggcaaaaaccacgccaactctgttcgagaatgatgtaatcaagaaggtctcacaaaaccatccaggcagtaccacttcccaagtattgcttagatgggcaactcagagaggcattgccgtcattccaaaatcttccaagaaggaaaggttacttggcaacctagaaatcgaaaaaaagttcactttaacggagcaagaattgaaggatatttctgcactaaatgccaacatcagatttaatgatccatggacctggttggatggtaaattccccacttttgcctga

SEQ ID NO: 24 - DNA, 828 bp, Artificial Sequence
  Escherichia coli str. K-12 substr. DH10B
atggctaatccaaccgttattaagctacaggatggcaatgtcatgccccagctgggactgggcgtctggcaagcaagtaatgaggaagtaatcaccgccattcaaaaagcgttagaagtgggttatcgctcgattgataccgccgcggcctacaagaacgaagaaggtgtcggcaaagccctgaaaaatgcctcagtcaacagagaagaactgttcatcaccactaagctgtggaacgacgaccacaagcgcccccgcgaagccctgctcgacagcctgaaaaaactccagcttgattatatcgacctctacttaatgcactggcccgttcccgctatcgaccattatgtcgaagcatggaaaggcatgatcgaattgcaaaaagagggattaatcaaaagcatcggcgtgtgcaacttccagatccatcacctgcaacgcctgattgatgaaactggcgtgacgcctgtgataaaccagatcgaacttcatccgctgatgcaacaacgccagctacacgcctggaacgcgacacacaaaatccagaccgaatcctggagcccattagcgcaaggagggaaaggcgttttcgatcagaaagtcattcgcgatctggcagataaatacggcaaaaccccggcgcagattgttatccgctggcatctggatagcggcctggtggtgatcccgaaatcggtcacaccttcacgtattgccgaaaactttgatgtctgggatttccgtctcgacaaagacgaactcggcgaaattgcaaaactcgatcagggcaagcgtctcggtcccgatcctgaccagttcggcggctaa

SEQ ID NO: 25 - DNA, 804 bp, Artificial Sequence
  Escherichia coli str. K-12 substr. DH10B
atggctatccctgcatttggtttaggtactttccgtctgaaagacgacgttgttatttcatctgtgataacggcgcttgaacttggttatcgcgcaattgataccgcacaaatctatgataacgaagccgcagtaggtcaggcgattgcagaaagtggcgtgccacgtcatgaactctacatcaccactaaaatctggattgaaaatctcagcaaagacaaattgatcccaagtctgaaagagagcctgcaaaaattgcgtaccgattatgttgatctgacgctaatccactggccgtcaccaaacgatgaagtctctgttgaagagtttatgcaggcgctgctggaagccaaaaaacaagggctgacgcgtgagatcggtatttccaacttcacgatcccgttgatggaaaaagcgattgctgctgttggtgctgaaaacatcgctactaaccagattgaactctctccttatctgcaaaaccgtaaagtggttgcctgggctaaacagcacggcatccatattacttcctatatgacgctggcgtatggtaaggccctgaaagatgaggttattgctcgtatcgcagctaaacacaatgcgactccggcacaagtgattctggcgtgggctatgggggaaggttactcagtaattccttcttctactaaacgtaaaaacctggaaagtaatcttaaggcacaaaatttacagcttgatgccgaagataaaaaagcgatcgccgcactggattgcaacgaccgcctggttagcccggaaggtctggctcctgaatgggattaa

SEQ ID NO: 26 - DNA, 939 bp, Artificial Sequence
  Saccharomyces cerevisiae S288c
atgcctgctactttacatgattctacgaaaatcctttctctaaatactggagcccaaatccctcaaataggtttaggtacgtggcagtcgaaagagaacgatgcttataaggctgttttaaccgctttgaaagatggctaccgacacattgatactgctgctatttaccgtaatgaagaccaagtcggtcaagccatcaaggattcaggtgttcctcgggaagaaatctttgttactacaaagttatggtgtacacaacaccacgaacctgaagtagcgctggatcaatcactaaagaggttaggattggactacgtagacttatatttgatgcattggcctgccagattagatccagcctacatcaaaaatgaagacatcttgagtgtgccaacaaagaaggatggttctcgtgcagtggatatcaccaattggaatttcatcaaaacctgggaattaatgcaggaactaccaaagactggtaaaactaaggccgttggagtctccaacttttctataaataacctgaaagatctattagcatctcaaggtaataagcttacgccagctgctaaccaagtcgaaatacatccattactacctcaagacgaattgattaatttttgtaaaagtaaaggcattgtggttgaagcttattctccgttaggtagtaccgatgctccactattgaaggaaccggttatccttgaaattgcgaagaaaaataacgttcaacccggacacgttgttattagctggcacgtccaaagaggttatgttgtcttgccaaaatctgtgaatcccgatcgaatcaaaacgaacaggaaaatatttactttgtctactgaggactttgaagctatcaataacatatcgaaggaaaagggcgaaaaaagggttgtacatccaaattggtctcctttcgaagtattcaagtaa

(19) United States
(12) Patent Application Publication
(10) Pub. No.: US 2013/0289276 A1
(43) Pub. Date: Oct. 31, 2013

(54) PROCESS FOR PREPARING AN INTERMEDIATE OF SITAGLIPTIN VIA ENZYMATIC CONVERSION

(71) Applicants: Sanjeev Kumar Mendirata, Ahmedabad (IN); Bipin Pandey, Ahmedabad (IN); Rupal Joshi, Ahmedabad (IN); Umang Trivedi, Ahmedabad (IN); Mayank G. Dave, Ahmedabad (IN); Himanshu M. Kothari, Ahmedabad (IN); Bhavin Shukla, Ahmedabad (IN)

(72) Inventors: Sanjeev Kumar Mendirata, Ahmedabad (IN); Bipin Pandey, Ahmedabad (IN); Rupal Joshi, Ahmedabad (IN); Umang Trivedi, Ahmedabad (IN); Mayank G. Dave, Ahmedabad (IN); Himanshu M. Kothari, Ahmedabad (IN); Bhavin Shukla, Ahmedabad (IN)

(73) Assignee: CADILA HEALTHCARE LIMITED, Ahmedabad (IN)

(21) Appl. No.: 13/823,300

(22) PCT Filed: Oct. 10, 2011
(86) PCT No.: PCT/IN2011/000702

§ 371 Date: Mar. 14, 2013

(30) Foreign Application Priority Data
Oct. 8, 2010 (IN) 2805/MUM/2010

(51) Int. Cl. C07D 487/04

(52) U.S. Cl. CPC C07D 487/04
USPC 544/350; 435/119; 435/320.1

(57) ABSTRACT

The invention provides a process for preparing 3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one (Formula I), into its racemic (R/S) form or any of its optically active (S) or (R) forms or enantiomeric excess mixture of any of the forms comprising:
a) reacting 4-oxo-4-[3-(trifluoromethyl)-5,6-dihydro[1,2,4]triazolo[4,3-a]pyrazin-7(8H)-yl]-1-(2,4,5-trifluorophenyl)butan-2-one of formula (III) with a suitable oxidoreductase enzymes or its suitable variants in the presence of suitable conditions and co-factor; and
b) isolating 3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one, into its racemic (R/S) form or any of its optically active (S) or (R) forms or enantiomeric excess mixture of any of the forms.

### FIELD OF THE INVENTION

The invention relates to the enzymatic reduction process for the preparation of 3-hydroxy-1-(3-(tri fluoromethyl)-5,6-dihydro-[1,2,4]triazolo[4,3-a]pyrazin-7(8H)-yl)-4-(2,4,5-trifluorophenyl)butan-1-one. In particular, the invention is directed to the stereoselective enzymatic reduction process for the preparation of (S) or (R)-3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one. The invention provides amino acid sequences of the polypeptides having relevant oxidoreductase activity. Furthermore the invention provides polynucleotide sequences encoding the polypeptides having oxidoreductase activity. The present invention also discloses cofactor regeneration system through substrate based or enzyme based system to regenerate the cofactor during the enzymatic reduction of interest.

### BACKGROUND OF THE INVENTION

3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one of the following formula (I),

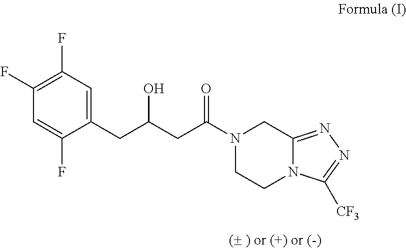 [SMILES: O=C(CC(O)Cc1cc(F)c(F)cc1F)N1CCn2c(nnc2C(F)(F)F)C1]

is a key intermediate for making the compound of formula (II), an industrially useful compound having the chemical name (2R)-4-oxo-4-[3-(trifluoromethyl)-5,6-dihydro[1,2,4]-triazolo[4,3-a]pyrazin-7(8H)-yl]-1-(2,4,5-trifluorophenyl)butan-2-amine (Sitagliptin).

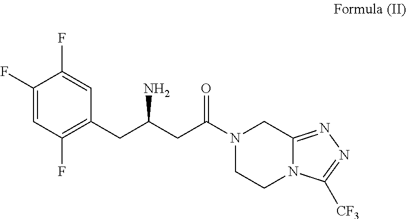 [SMILES: N[C@@H](CC(=O)N1CCn2c(nnc2C(F)(F)F)C1)Cc1cc(F)c(F)cc1F]

WO 03004498 and U.S. Pat. No. 6,699,871 both assigned to Merck & Co., describe a class of beta-amino tetrahydrotriazolo[4,3-a]pyrazines, which are inhibitors of DPP-IV. Disclosed therein are compounds, whose general formula is,

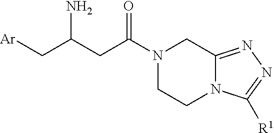

Specifically disclosed in WO 03004498 is (2R)-4-oxo-4-[3-(trifluoromethyl)-5,6-dihydro[1,2,4]-triazolo[4,3-a]pyrazin-7(8H)-yl]-1-(2,4,5-trifluorophenyl)butan-2-amine (Sitagliptin).

PCT Publication NO. WO2010032264 (WO' 264) disclosed the compound 3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one. WO'264 also refers to process for the preparation of the 3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one by using chemical reduction method, the reduction is performed by a suitable borane containing reducing agent, in absence or presence of an acid in a suitable solvent to obtain 3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one. The process is illustrated in scheme 1 below:

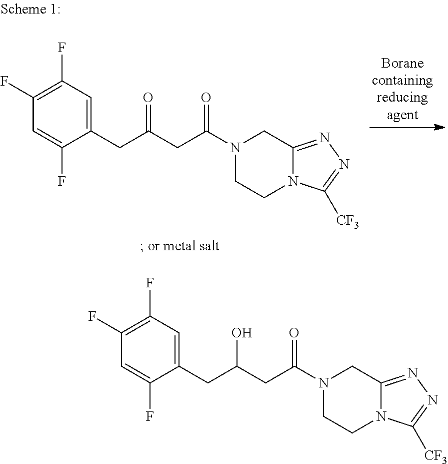 [SMILES: O=C(CC(=O)N1CCn2c(nnc2C(F)(F)F)C1)Cc1cc(F)c(F)cc1F ; O=C(CC(O)Cc1cc(F)c(F)cc1F)N1CCn2c(nnc2C(F)(F)F)C1]

Moreover, WO'264 only provides the racemate form of 3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one (Formula I) and no chemical process is reported to prepare the active R or S form from the racemate of formula (I).

In addition, WO2010032264 describes the use of metal catalysts which leave trace amounts of the metal in the final product and which are problematic for the manufacture of pharmaceutical products.

Therefore, the chemical processes are not as efficient to prepare the compound of formula (I) at low cost as they consume expensive solvents and other chemicals which additionally are difficult to handle at large scale and moreover these are not environment friendly.

Moreover, one of the major drawbacks of the chemical procedures is that during resolution step, theoretically only 50% of the total material can be isolated from the racemic mixture as a pure enantiomer. Thus wastage of 50% unwanted material makes the procedure costly and has an adverse effect on the environment. Also recycling of the wrong isomer requires extra unit operations and cost.

Hence there is a high unmet need to develop a process for the resolution of compound of formula (I) to its optically active, R and S form, at low cost and which should be environment friendly.

With the advent of biotechnology, it has been possible to develop enzymatic processes to obtain enantiomerically pure compound. Enzymes can have a unique stereo selective property of producing only one enantiomer with good chiral purity.

The enzymatic reduction processes of the invention in which the enzyme acts as a reduction catalyst are environmentally advantageous compared to the use of metal catalysts as described in the prior art. The use of the enzymes is also typically lower in cost than the processes using the catalyst as in WO2010032264.

We herein disclose a process for the preparation of compound formula (I), in racemic (R/S) form or any of its optically active, (S) or (R) forms or as an enantiomeric excess mixture of any of the forms by using enzymatic reduction. We herein also disclose (S) and (R) enantiomer of 3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one in high enantiomeric purity.

Disclosed herein are also processes for preparing the (R) & (S) forms of compound of formula (I) through stereoselective enzymatic reduction of the corresponding keto compound.

### SUMMARY OF THE INVENTION

The present invention provides a process for the preparation of suitable intermediate of formula (I)

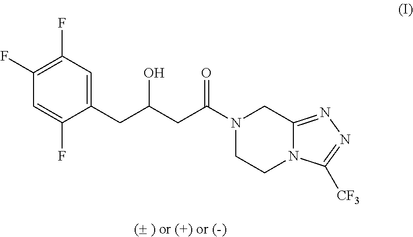 [SMILES: O=C(CC(O)Cc1cc(F)c(F)cc1F)N1CCn2c(nnc2C(F)(F)F)C1]

comprising:
a) reacting 4-oxo-4-[3-(trifluoromethyl)-5,6-dihydro[1,2,4]triazolo[4,3-a]pyrazin-7(8H)-yl]-1-(2,4,5-trifluorophenyl)butan-2-one of formula (III) or its metal ion salt:

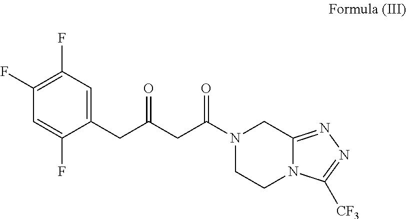 [SMILES: O=C(CC(=O)N1CCn2c(nnc2C(F)(F)F)C1)Cc1cc(F)c(F)cc1F]

with a suitable enzyme and variant thereof that stereoselectively reduces a ketone to form an alcohol, by maintaining under suitable conditions and cofactor
b) isolating the suitable intermediate

In one embodiment, the invention provides (S)-3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one.

In one embodiment, the invention provides (R)-3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one.

In one embodiment, the invention provides (S)-3-(methanesulfonyloxy)-1-(3-(trifluoromethyl)-5,6-dihydro-[1,2,4]triazolo[4,3-a]pyrazin-7(8H)-yl)-4-(2,4,5-trifluorophenyl)butan-1-one.

In one embodiment, the invention provides (R)-3-(methanesulfonyloxy)-1-(3-(trifluoromethyl)-5,6-dihydro-[1,2,4]triazolo[4,3-a]pyrazin-7(8H)-yl)-4-(2,4,5-trifluorophenyl)butan-1-one.

In one embodiment, the invention provides (S)-3-azido-1-(3-(trifluoromethyl)-5,6-dihydro-[1,2,4]triazolo[4,3-a]pyrazin-7(8H)-yl)-4-(2,4,5-trifluorophenyl)butan-1-one.

In one embodiment, the invention provides (R)-3-azido-1-(3-(trifluoromethyl)-5,6-dihydro-[1,2,4]triazolo[4,3-a]pyrazin-7(8H)-yl)-4-(2,4,5-trifluorophenyl)butan-1-one.

In one embodiment, the present invention provides a process for preparing 3-hydroxy-1-(3-(tri fluoromethyl)-5,6-dihydro-[1,2,4]triazolo[4,3-a]pyrazin-7(8H)-yl)-4-(2,4,5-trifluorophenyl)butan-1-one (Formula I), into its racemic (R/S) form or any of its optically active (S) or (R) forms or enantiomeric excess mixture of any of the forms to comprising: a) reacting 4-oxo-4-[3-(trifluoromethyl)-5,6-dihydro[1,2,4]triazolo[4,3-a]pyrazin-7(8H)-yl]-1-(2,4,5-trifluorophenyl)butan-2-one of formula (III) or its metal ion salt:

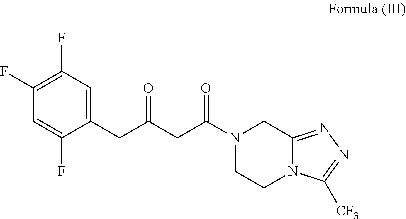 [SMILES: O=C(CC(=O)N1CCn2c(nnc2C(F)(F)F)C1)Cc1cc(F)c(F)cc1F]

with a suitable enzyme and variant thereof that stereoselectively reduces a ketone to form an alcohol, by maintaining under suitable conditions, to obtain 3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one, into its racemic (R/S) form or any of its optically active (S) or (R) forms or enantiomeric excess mixture of any of the forms.

In one embodiment, the present invention provides stereoselective enzymatic reduction processes for the preparation of 3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one, a key intermediate in the synthesis of 4-oxo-4-[3-(trifluoromethyl)-5,6-dihydro[1,2,4]-triazolo[4,3-a]pyrazin-7(8H)-yl]-1-(2,4,5-trifluorophenyl)butan-2-amine, in racemic (R/S) form or any of its optically active (S) or (R) forms, in high enantiomeric purity.

In one embodiment, present invention provides a process for preparing 3-hydroxy-1-(3-(tri fluoromethyl)-5,6-dihydro-[1,2,4]triazolo[4,3-a]pyrazin-7(8H)-yl)-4-(2,4,5-trifluorophenyl)butan-1-one, in racemic (R/S) form or any of its optically active (S) or (R) forms comprising reacting 4-oxo-4-[3-(trifluoromethyl)-5,6-dihydro[1,2,4]triazolo[4,3-a]pyrazin-7(8H)-yl]-1-(2,4,5-trifluorophenyl)butan-2-one of formula (III) with a suitable enzyme and their variants, optionally with external co-factor(s) and maintaining the solution, preferably with stirring, for a time sufficient to convert 3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one [Formula (I)], into its racemic (R/S) form or any of its optically active (S) or (R) forms or enantiomeric excess mixture of any of the forms, by enzymatic reduction.

In one embodiment, present invention provides the (R)-enantiomer of 3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one.

In another embodiment, present invention provides the (S)-enantiomer 3-hydroxy-1-(3-(tri fluoromethyl)-5,6-dihydro-[1,2,4]triazolo[4,3-a]pyrazin-7(8H)-yl)-4-(2,4,5-trifluorophenyl)butan-1-one.

In one embodiment, present invention provides a process for preparing Sitagliptin.

The process comprises converting the (S)-3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one or their enantiomerically excess mixtures into, (S)-3-(methanesulfonyloxy)-1-(3-(trifluoromethyl)-5,6-dihydro-[1,2,4]triazolo[4,3-a]pyrazin-7(8H)-yl)-4-(2,4,5-trifluorophenyl)butan-1-one; which can be further converted to ((R)-3-azido-1-[3-(trifluoromethyl)-5,6-dihydro[1,2,4]triazolo[4,3-a]pyrazin-7(8H)-yl]-4-(2,4,5-trifluorophenyl)butan-1-one; which is finally converted to (R)-4-oxo-4-[3-(tri fluoromethyl)-5,6-dihydro[1,2,4]triazolo[4,3-a]pyrazin-7(8H)-yl]-1-(2,4,5-trifluorophenyl)butan-2-amine (Sitagliptin).

In one embodiment, the present invention provides a process for preparing Sitagliptin. The process comprises converting the optically pure, 3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one obtained as above, into Sitagliptin.

In an embodiment of the present invention is provided the amino acid sequences of the enzymes used in this invention.

In another embodiment of the present invention is provided the nucleotide sequences of the enzymes used in this invention.

In yet another embodiment of the present invention are provided the oxidoreductase enzyme and amino acid and nucleotide sequences thereof derived from species of Saccharomyces, Pyrococcus, Cupriavidus, Rhodotorula, Pichia and E. coli

In a further embodiment of the present invention is provided an expression vector comprising gene encoding the desired polypeptide having oxidoreductase enzymatic activity.

In yet another embodiment of the present invention is provided a polycistronic expression vector comprising a polynucleotide sequence encoding a polypeptide having oxidoreductase activity and another polynucleotide sequence encoding the second polypeptide having the enzymatic potential to generate reduced co-factor from oxidized cofactor e.g., NAD(P)H from NAD(P).

Accordingly, in embodiment it is an object of the invention to provide a method for co-expressing an oxidoreductase enzyme and a polypeptide having the enzymatic potential to generate reduced co-factor.

In yet another embodiment of the present invention are provided co-factor regenerative systems selected from substrate coupled or enzyme coupled systems.

A further embodiment of the present invention provides a process for the production of 3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one, in its racemic (R/S) form or any of its optically active (S) or (R) forms or enantiomeric excess mixture of any of the forms by reduction of 4-oxo-4-[3-(trifluoromethyl)-5,6-dihydro[1,2,4]triazolo[4,3-a]pyrazin-7(8H)-yl]-1-(2,4,5-trifluorophenyl)butan-2-one of formula (III) or its metal ion salt in the presence of oxidoreductase enzyme derived from Saccharomyces cerevisiae, Pyrococcus furiosus Rhodotorula mucilaginosa, Cupriavidus necator, Pichia methanolica and E. coli.

In a still further embodiment of the present invention is provided a process of production of 3,3-hydroxy-1-(3-(tri fluoromethyl)-5,6-dihydro-[1,2,4]triazolo[4,3-a]pyrazin-7(8H)-yl)-4-(2,4,5-trifluorophenyl)butan-1-one, in its racemic (R/S) form or any of its optically active (S) or (R) forms or enantiomeric excess mixture of any of the forms by reduction of 4-oxo-4-[3-(trifluoromethyl)-5,6-dihydro[1,2,4]triazolo[4,3-a]pyrazin-7(8H)-yl]-1-(2,4,5-trifluorophenyl)butan-2-one of formula (III) or its metal ion salt using whole cell biocatalysis. In such embodiment the whole cell is selected from MTCC 5642, MTCC 5643, MTCC 5644, MTCC 5645, MTCC 5646, MTCC 5647, MTCC 5648, MTCC 5649, MTCC 5650, MTCC 5651, MTCC 5652, MTCC 5653, and MTCC 5654.

In yet another embodiment of the present invention is provided the over-expression of the desired polypeptide having the desired oxidoreductase enzymatic activity in E. coli transformed cells.

In another embodiment, the invention provides (S)-3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one.

In one embodiment, the invention provides (R)-3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one.

In one embodiment, the invention provides (S)-3-(methanesulfonyloxy)-1-(3-(trifluoromethyl)-5,6-dihydro-[1,2,4]triazolo[4,3-a]pyrazin-7(8H)-yl)-4-(2,4,5-trifluorophenyl)butan-1-one.

In one embodiment, the invention provides (R)-3-(methanesulfonyloxy)-1-(3-(trifluoromethyl)-5,6-dihydro-[1,2,4]triazolo[4,3-a]pyrazin-7(8H)-yl)-4-(2,4,5-trifluorophenyl)butan-1-one.

In one embodiment, the invention provides (S)-3-azido-1-(3-(trifluoromethyl)-5,6-dihydro-[1,2,4]triazolo[4,3-a]pyrazin-7(8H)-yl)-4-(2,4,5-trifluorophenyl) butan-1-one.

In one embodiment, the invention provides (R)-3-azido-1-(3-(trifluoromethyl)-5,6-dihydro-[1,2,4]triazolo[4,3-a]pyrazin-7(8H)-yl)-4-(2,4,5-trifluorophenyl)butan-1-one.

In embodiment the invention provides a process for preparing 3-hydroxy-1-(3-(tri fluoromethyl)-5,6-dihydro-[1,2,4]triazolo[4,3-a]pyrazin-7(8H)-yl)-4-(2,4,5-trifluorophenyl)butan-1-one (Formula I), in its racemic (R/S) form or any of its optically active (S) or (R) forms or enantiomeric excess mixture of any of the forms comprising: a) reacting 4-oxo-4-[3-(trifluoromethyl)-5,6-dihydro[1,2,4]triazolo[4,3-a]pyrazin-7(8H)-yl]-1-(2,4,5-trifluorophenyl)butan-2-one of formula (III) or its metal ion salt:

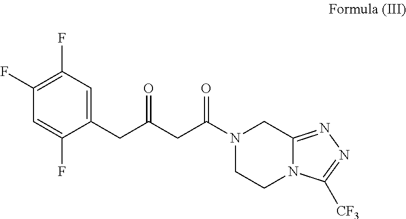 [SMILES: O=C(CC(=O)N1CCn2c(nnc2C(F)(F)F)C1)Cc1cc(F)c(F)cc1F]

with a suitable oxidoreductase enzyme or its suitable variant in the presence of suitable conditions and co-factor.
b) isolating 3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one, in its racemic (R/S) form or any of its optically active (S) or (R) forms or enantiomeric excess mixture of any of the forms.

In embodiment, the present enzyme works in the presence of cofactor NAD(P) where the cofactor is regenerated by substrate coupled or enzyme coupled system. The present invention also provides recombinant vectors either only containing genes coding for suitable polypeptides with oxido-reductase activity or those additionally containing gene encoding a polypeptide having the capacity to enzymatically regenerate the co-factor. The said vector is transformed in suitable host cell.

In one embodiment, present invention provides a process for preparing Sitagliptin.

The process comprises converting the (S)-3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one or their enantiomerically excess mixtures into (S)-3-(methanesulfonyloxy)-1-(3-(trifluoromethyl)-5,6-dihydro-[1,2,4]triazolo[4,3-a]pyrazin-7(8H)-yl)-4-(2,4,5-trifluorophenyl)butan-1-one; which can be further converted to ((R)-3-azido-1-[3-(trifluoromethyl)-5,6-dihydro[1,2,4]triazolo[4,3-a]pyrazin-7(8H)-yl]-4-(2,4,5-trifluorophenyl)butan-1-one; which is finally converted to (R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine (Sitagliptin).

### BRIEF DESCRIPTION OF DRAWING

FIG. 1 depicts pET11a oxidoreductase [Seq Id no 1, 2, 3, 4, 5 and 7]

FIG. 2 depicts pET27b oxidoreductase [Seq Id no 1, 3, 5, 6, 7, 8, 9, 10, 11, 12, 13]

FIG. 3 depicts pZRC2G-2 oxidoreductase

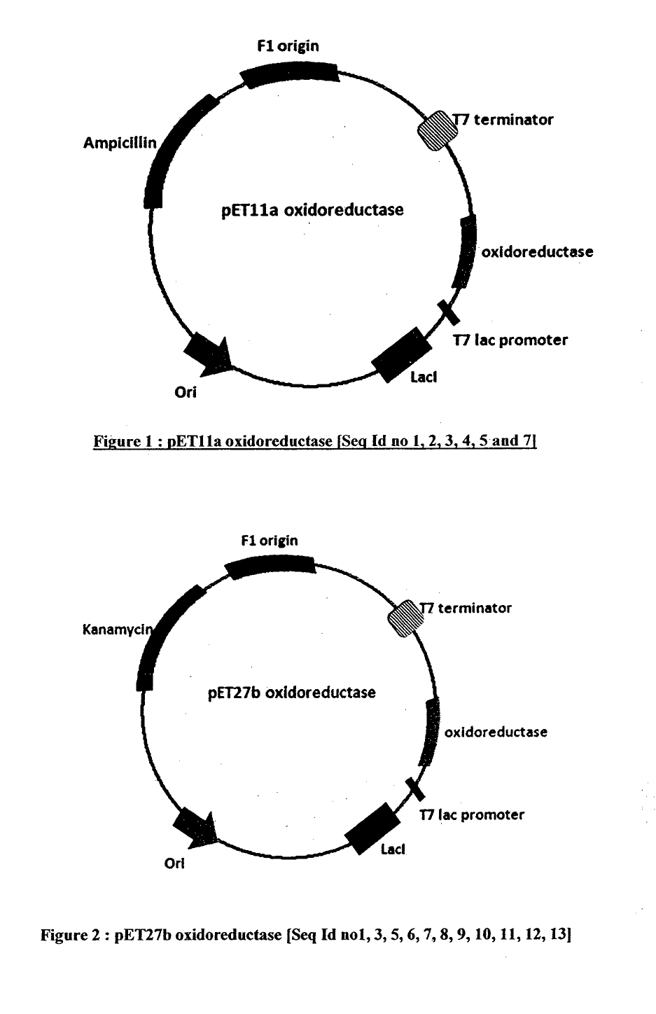

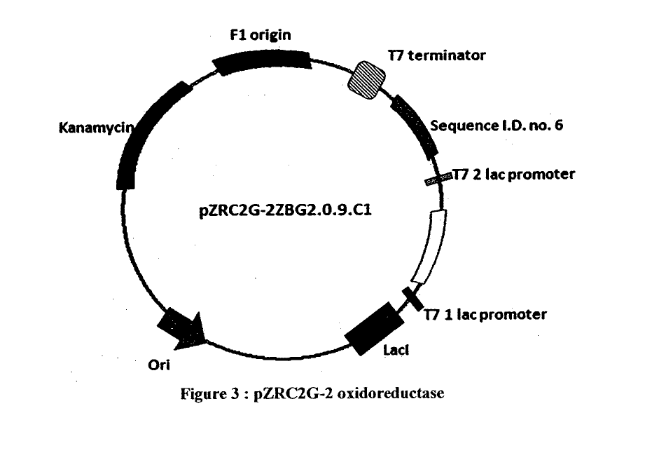

### DETAILED DESCRIPTION OF THE INVENTION

The Amino Acid Sequences Ids 1 to 13 and their corresponding nucleotide sequences Ids 14 to 26 are depicted below. Reference to any of the amino acid sequences by their Ids 1 to 13 will also deemed to include their corresponding Nucleotide sequence by their Ids 14 to 26.

SEQUENCES

Sequence Id No. 1
Amino acid Sequence
MKRVNAFNDLKRIGDDKVTAIGMGTWGIGGRETPDYSRDKESIEAIRYGLELG
MNLIDTAEFYGAGHAEEIVGEAIKEFEREDIFIVSKVWPTHFGYEEAKKAARAS
AKRLGTYIDLYLLHWPVDDFKKIEETLHALEDLVDEGVIRYIGVSNFNLELLQR
SQEVMRKYEIVANQVKYSVKDRWPETTGLLDYMKREGIALMAYTPLEKGTLA
RNECLAKIGEKYGKTAAQVALNYLIWEENVVAIPKASNKEHLKENFGAMGWR
LSEEDREMARRCV
Sequence ID 14 (corresponding to Sequence ID 1)
DNA Sequence
ATGAGGCCAGTTAATTAAGAGGTACCATATGAAACGCGTGAATGCCTTTAA
TGATCTGAAACGCATTGGTGATGATAAAGTTACCGCAATTGGTATGGGCAC
CTGGGGTATTGGTGGTCGTGAAACACCGGATTATAGCCGTGATAAAGAAAG
CATTGAAGCCATTCGTATTGGTGGTCGTGAAACACCGGATTATAGCCGTGA
TAAAGAAAGCATTGAAGCCATTCGTTATGGTCTGGAACTGGGCATGAATCT
GATTGATACCGCAGAATTTTATGGTGCAGGCCATGCAGAAGAAATTGTTGG
CGAAGCCATCAAAGAATTTGAACGCGAGGATATCTTTATTGTTAGCAAAGT
GTGGCCGACCCATTTTGGTTATGAAGAAGCCAAAAAAGCAGCACGTGCAA
GTTATATTGGCGTGAGCAACTTTAATCTGGAACTGCTGCAGCGTAGCCAAG
AAGTTATGCGCAAATACGAAATTGTTGCCAACCAGGTGAAATATAGCGTTA
AAGATCGTTGGCCTGAAACCACCGGTCTGCTGGATTATATGAAACGTGAAG
GTATTGCACTGATGGCATATACACCGCTGGAAAAAGGCACCCTGGCACGTA
ATGAATGTCTGGCCAAAATTGGCGAAAAATATGGTAAAACCGCAGCACAG
GTTGCACTGAATTATCTGATCTGGGAAGAAAATGTTGTTGCAATTCCGAAA
GCCAGCAACAAAGAACATCTGAAAGAAAATTTTGGTGCAATGGGTTGGCGT
CTGAGCGAAGAGGATCGTGAAATGGCACGTCGTTGTGTTTAA
Sequence Id No. 2
Amino acid Sequence
MNWEKVPQELYTRLGSSGLQISKIIVGCMSFGTKAWGGDWVLEDEDEIFAIMK
KAYDQGIRTFDTADSYSNGVSERLLGKFIRKYNIDRSKLVILTKVFFPAPEEYES
FSFFNHNFPGHELVNRSGLSRKHILDSAAASVERLGTYIDVLQIHRYDPNTPAEE
TMEALNDCIKQGLTRYIGASTMRAYQFIKYQNVAEKHGWAKFISMQSYYSLL
YREEEAELIAYCNETGVGLIPWSPNAGGFLTRPVSKQDTARSASGAAALYGLEP
FSEADKAIIDRVEELSKKKGVSMASVALAWVISKNSWPIIGFSKPGRVDDALDG
FKLKLTEEDIKFLEEPYVPKPLPRLYSVIL
Sequence ID 15 (corresponding to Sequence ID 2)
DNA Sequence
ATGAGGCCAGTTAATTAAGAGGTACCATATGAATTGGGAAAAAGTGCCGCA
GGAACTGTATACCCGTCTGGGTAGCAGCGGTCTGCAGATTAGCAAAATTAT
TGTGGGTTGTATGAGCTTTGGCACCAAAGCATGGGGTGGTGATTGGGTTCT
GGAAGATGAAGATGAAATTTTTGCCATTATGAAAAAAGCCTATGATCAGGG
TATTCGTACCTTTGATACCGCAGATAGCTATAGCAATGGTGTTAGCGAACGT
CTGCTGGGTAAATTCATCCGCAAATACAACATTGATCGCAGCAAACTGGTT
ATTCTGACCAAAGTTTTTTTTCCGGCACCGGAAGAATATGAAAGCTTCAGCT
TTTTTAACCATAACTTTCCGGGTCATGAACTGGTTAATCGTAGCGGTCTGAG
CCGTAAACATATTCTGGATAGCGCAGCAGCAAGCGTTGAACGTCTGGGCAC
CTATATTGATGTTCTGCAGATCCATCGTTATGATCCGAATACACCGGCTGAA
GAAACAATGGAAGCCCTGAACGATTGTATTAAACAGGGTCTGACCCGTTAT
ATTGGTGCAAGCACCATGCGTGCCTATCAGTTCATTAAATATCAGAACGTG
GCCGAAAAACATGGTTGGGCCAAATTTATTAGCATGCAGAGCTATTATAGC
CTGCTGTATCGTGAAGAAGAAGCAGAACTGATTGCCTATTGCAATGAAACC
GGTGTTGGTCTGATTCCGTGGAGCCCGAATGCCGGTGGTTTTCTGACCCGTC
CGGTTAGCAAACAGGATACCGCACGTAGCGCAAGCGGTGCAGCAGCACTG
TATGGTCTGGAACCGTTTAGCGAAGCAGATAAAGCCATTATTGATCGTGTG
GAAGAACTGAGCAAAAAAAAAGGTGTTAGCATGGCAAGCGTTGCACTGGC
ATGGGTTATTAGCAAAAACAGCTGGCCGATTATTGGTTTTAGCAAACCGGG
TCGTGTTGATGATGCACTGGATGGCTTTAAACTGAAACTGACCGAAGAGGA
TATCAAATTCCTGGAAGAACCGTATGTTCCGAAACCGCTGCCTCGTCTGTAT
AGCGTTATTCTGTAA
Sequence Id No. 3
Amino acid Sequence
MSQGRKAAERLAKKTVLITGASAGIGKATALEYLEASNGDMKLILAARRLEKL
EELKKTIDQEFPNAKVHVAQLDITQAEKIKPFIENLPQEFKDIDILVNNAGKALG
SDRVGQIATEDIQDVFDTNVTALINITQAVLPIFQAKNSGDIVNLGSIAGRDAYP
TGSIYCASKFAVGAFTDSLRKELINTKIRVILIAPGLVETEFSLVRYRGNEEQAK
NVYKDTTPLMADDVADLIVYATSRKQNTVIADTLIFPTNQASPHHIFRG
Sequence ID 16 (corresponding to Sequence ID 3)
DNA Sequence
ATGAGGCCAGTTAATTAAGAGGTACCATATGAGCCAGGGTCGTAAAGCAGC
AGAACGTCTGGCAAAAAAAACCGTTCTGATTACCGGTGCAAGCGCAGGTAT
TGGTAAAGCAACCGCACTGGAATATCTGGAAGCAAGCAATGGCGATATGA
AACTGATTCTGGCAGCACGTCGTCTGGAAAAACTGGAAGAACTGAAAAAA
ACCATCGATCAGGAATTTCCGAACGCAAAAGTTCATGTTGCACAGCTGGAT
ATTACCCAGGCAGAAAAAATCAAACCGTTTATCGAAAATCTGCCGCAGGAA
TTCAAAGATATCGATATTCTGGTGAATAATGCAGGTAAAGCACTGGGTAGC
GATCGTGTTGGTCAGATTGCAACCGAAGATATCCAGGATGTGTTTGATACC
AATGTGACCGCACTGATTAATATTACACAGGCCGTTCTGCCGATTTTTCAGG
CAAAAAACAGCGGTGATATTGTGAATCTGGGTAGCATTGCAGGTCGTGATG
CATATCCGACCGGTAGCATTTATTGTGCAAGCAAATTTGCAGTTGGTGCATT
TACCGACAGTCTGCGCAAAGAACTGATTAATACCAAAATCCGCGTTATTCT
GATTGCACCGGGTCTGGTTGAAACCGAATTCAGCCTGGTTCGTTATCGTGGT
AATGAAGAACAGGCCAAAAACGTGTATAAAGATACCACACCGCTGATGGC
AGATGATGTTGCCGATCTGATTGTTTATGCAACCAGCCGTAAACAGAATAC
CGTTATTGCCGATACCCTGATTTTTCCGACCAATCAGGCATCTCCGCATCAT
ATTTTTCGTGGTTAA
Sequence Id No. 4
Amino acid Sequence
MTQRIAYVTGGMGGIGTAICQRLAKDGFRVVAGCGPNSPRREKWLEQQKALG
FDFIASEGNVADWDSTKTAFDKVKSEVGEVDVLINNAGITRDVVFRKMTRAD
WDAVIDTNLTSLFNVTKQVIDGMADRGWGRIVNISSVNGQKGQFGQTNYSTA
KAGLHGFTMALAQEVATKGVTVNTVSPGYIATDMVKAIRQDVLDKIVATIPVK
RLGLPEEIASICAWLSSEESGFSTGADFSLNGGLHMG
Sequence ID 17 (corresponding to Sequence ID 4)
DNA Sequence
ATGAGGCCAGTTAATTAAGAGGTACCATATGACCCAGCGTATTGCCTATGT
TACCGGTGGTATGGGTGGTATTGGCACCGCAATTTGTCAGCGTCTGGCAAA
AGATGGTTTTCGTGTTGTTGCAGGTTGTGGTCCGAATTCTCCGCGTCGTGAA
AAATGGCTGGAACAGCAGAAAGCACTGGGTTTTGATTTTATTGCCAGCGAA
GGTAATGTTGCAGATTGGGATAGCACCAAAACCGCCTTTGATAAAGTTAAA
AGCGAAGTGGGTGAAGTTGATGTGCTGATTAACAATGCAGGTATTACCCGT
GATGTTGTGTTTCGCAAAATGACCCGTGCCGATTGGGATGCAGTTATTGATA
CCAATCTGACCAGCCTGTTTAATGTTACCAAACAGGTGATTGATGGTATGG
CAGATCGTGGTTGGGGTCGTATTGTTAATATTAGCAGCGTGAATGGTCAGA
AAGGTCAGTTTGGTCAGACCAATTATAGCACCGCAAAAGCAGGTCTGCATG
GTTTTACAATGGCACTGGCACAGGAAGTTGCAACCAAAGGCGTTACCGTTA
ATACCGTTTCTCCGGGTTATATTGCCACCGATATGGTTAAAGCAATTCGTCA
GGATGTGCTGGATAAAATTGTTGCCACCATTCCGGTTAAACGTCTGGGTCTG
CCGGAAGAAATTGCAAGCATTTGTGCATGGCTGAGCAGCGAAGAAAGCGG
TTTTAGCACAGGTGCAGATTTTAGCCTGAATGGTGGTCTGCACATGGGTTAA
Sequence Id No. 5
Amino acid Sequence
MSSPSDGPFPKATPQLPNSVFDMFSMKGKVTAITGGGGGIGFAAAEAIAEAGG
DVALLYRSAPNMEERSAELAKRFGVKVKSYQCEVTEHESVKQAIEAVEKDFG
RLDCYIANAGGGVPGSINPDYPLEAWHKTQSVNLHSTFYAARECARIFKAQGS
GSFIATTSISARIVNVPYDQPAYNSSKAAVVHFCRSLARDWRNFARVNTISPGFF
DTPMGPSDKAVEDVLYQKSVLGRAGDVKELKAAYLYLASNASTYTTGADLLI
DGGYCLT
Sequence ID 18 (corresponding to Sequence ID 5)
DNA Sequence
ATGAGGCCAGTTAATTAAGAGGTACCATATGAGCAGCCCGTCTGATGGTCC
GTTTCCGAAAGCAACACCGCAGCTGCCGAATAGCGTTTTTGACATGTTTAG
CATGAAAGGTAAAGTTACCGCAATTACCGGTGGTGGTGGTGGCATTGGTTT
TGCAGCAGCAGAAGCAATTGCCGAAGCCGGTGGTGATGTTGCACTGCTGTA
TCGTAGCGCACCGAATATGGAAGAACGTAGCGCAGAACTGGCAAAACGTT
TTGGTGTGAAAGTGAAAAGCTATCAGTGCGAAGTTACCGAACATGAAAGCG
TTAAACAGGCAATTGAAGCCGTGGAAAAAGATTTTGGTCGCCTGGATTGTT
ATATTGCAAATGCGGGTGGTGGTGTTCCGGGTAGCATTAATCCGGATTATC
CGCTGGAAGCATGGCATAAAACCCAGAGCGTTAATCTGCATAGCACCTTTT
ATGCAGCACGTGAATGCGCACGTATTTTTAAAGCACAGGGCAGCGGTAGCT
TTATTGCAACCACCTCTATTAGCGCACGTATTGTGAATGTTCCGTATGATCA
GCCTGCATATAATAGCAGCAAAGCAGCCGTTGTTCATTTTTGTCGTAGCCTG
GCACGTGATTGGCGTAATTTTGCCCGTGTTAATACCATTAGCCCTGGTTTTT
TTGATACCCCGATGGGTCCGAGCGATAAAGCAGTTGAAGATGTGCTGTATC
AGAAAAGCGTTCTGGGTCGTGCCGGTGATGTTAAAGAACTGAAAGCAGCAT
ATCTGTATCTGGCAAGCAATGCAAGCACCTATACCACCGGTGCAGATCTGC
TGATTGATGGTGGTTATTGTCTGACCTAA
Sequence Id No. 6
Amino acid Sequence
MVPKFYKLSNGFKIPSIALGTYDIPRSQTAEIVYEGVKCGYRHFDTAVLYGNEK
EVGDGIIKWLNEDPGNHKREEIFYTTKLWNSQNGYKRAKAAIRQCLNEVSGLQ
YIDLLLIHSPLEGAVDEGLVKSIGVSNYGKKHIDELLNWPELKHKPVVNQIEISP
WIMRQELADYCKSKGLVVEAFAPLCHGYKMTNPDLLKVCKEVDRNPGQVLIR
WSLQHGYLPLPKTKTVKRLEGNLAAYNFELSDEQMKFLDHAP
Sequence ID 19 (corresponding to Sequence ID 6)
DNA Sequence
ATGGTTCCTAAGTTTTACAAACTTTCAAACGGCTTCAAAATCCCAAGCATTG
CTTTGGGAACCTACGATATTCCAAGATCGCAAACAGCCGAAATTGTGTATG
AAGGTGTCAAGTGCGGCTACCGTCATTTCGATACTGCTGTTCTTTATGGTAA
TGAGAAGGAAGTTGGCGATGGTATCATTAAATGGTTGAACGAAGATCCAGG
GAACCATAAACGTGAGGAAATCTTCTACACTACTAAATTATGGAATTCGCA
AAACGGATATAAAAGAGCTAAAGCTGCCATTCGGCAATGTTTGAATGAAGT
CTCGGGCTTGCAATACATCGATCTTCTTTTGATTCATTCGCCACTGGAAGGT
TCTAAATTAAGGTTGGAAACTTGGCGCGCCATGCAAGAAGCGGTTGATGAA
GGATTGGTTAAGTCTATAGGGGTTTCCAACTATGGGAAAAAGCACATTGAT
GAACTTTTGAACTGGCCAGAACTGAAGCACAAGCCAGTGGTCAACCAAATC
GAGATATCACCTTGGATTATGAGACAAGAATTAGCAGATTACTGTAAATCT
AAAGGTCTCGTCGTCGAAGCCTTTGCCCCATTGTGTCACGGCTACAAAATG
ACTAATCCAGATTTATTAAAAGTTTGCAAAGAGGTGGACCGTAATCCAGGT
CAAGTTTTGATTCGTTGGTCTTTACAACACGGTTATTTACCACTACCGAAGA
CTAAAACTGTGAAGAGGTTAGAAGGTAACCTTGCAGCCTACAACTTTGAAC
TGTCAGACGAACAGATGAAATTTCTTGATCATCCTGATGCTTATGAGCCTAC
CGATTGGGAATGCACAGACGCGCCATAA
Sequence Id No. 7
Amino acid Sequence
MYTDLKDKVVVVTGGSKGLGRAMAVRFGQEQSKVVVNYRSNEEEALEVKKE
IEQAGGQAIIVRGDVTKEEDVVNLVETAVKEFGTLDVMINNAGVENPVPSHEL
SLENWNQVIDTNLTGAFLGSREAIKYFVENDIKGNVINMSSVHEMIPWPLFVHY
AASKGGMKLMTETLALEYAPKGIRVNNIGPGAIDTPINAEKFADPEQRADVES
MIPMGYIGNPEEIASVAAFLASSQASYVTGITLFADGGMTKYPSFQAGRG
Sequence ID 20 (corresponding to Sequence ID 7)
DNA Sequence
ATGTATACCGACCTGAAAGATAAAGTTGTTGTTGTGACCGGTGGTAGCAAA
GGTCTGGGTCGTGCAATGGCAGTTCGTTTTGGTCAGGAACAGAGCAAAGTT
GTTGTGAATTATCGCAGCAATGAAGAAGAAGCCCTGGTTGGTCAGGAACAG
AGCAAAGTTGTTGTGAATTATCGCAGCAATGAAGAAGAAGCCCTGGCCAAA
GAAGAGGACGTTGTTAATCTGGTTGAAACCGCAGTTAAAGAATTTGGCACC
CTGGATGTGATGATTAATAATGCCGGTGTTGAAAATCCGGTTCCGAGCCAT
GAACTGAGCCTGGAAAATTGGAATCAGGTGATTGATACCAATCTGACCGGT
GCATTTCTGGGTAGCCGTGAAGCCATTAAATATTTTGTGGAAAATGATATTA
AAGGCAATGTGATCAATATGAGCAGCGTTCATGAAATGATTCCGTGGCCTC
TGTTTGTTCATTATGCAGCAAGCAAAGGTGGTATGAAACTGATGACCGAAA
CCCTGGCACTGGAATATGCACCGAAAGGTATTCGTGTGAATAATATTGGTC
CGGGTGCAATTGATACCCCGATCAATGCAGAAAAATTTGCAGATCCGGAAC
AGCGTGCAGATGTTGAAAGCATGATTCCGATGGGTTATATTGGCAATCCGG
AAGAAATTGCAAGCGTTGCAGCATTTCTGGCAAGCAGCCAGGCAAGCTATG
TTACCGGTATTACCCTGTTTGCAGATGGTGGTATGACCAAATATCCGAGCTT
TCAGGCAGGTCGTGGTTAATAA
Sequence Id No. 8
Amino acid Sequence
MTDLFKPLPEPPTELGRLRVLSKTAGIRVSPLILGGASIGDAWSGFMGSMNKEQ
AFELLDAFYEAGGNCIDTANSYQNEESEIWIGEWMASRKLRDQIVIATKFTGDY
KKYEVGGGKSANYCGNHKRSLHVSVRDSLRKLQTDWIDILYIHWWDYMSSIE
EVMDSLHILVQQGKVLYLGVSDTPAWVVSAANYYATSHGKTPFSVYQGKWN
VLNRDFERDIIPMARHFGMALAPWDVMGGGRFQSKKAMEERKKNGEGLRTF
VGGPEKIAEEHGTESVTAIAIAYVRSKAKNVFPLIGGRKIEHLKQNIEALSIKLTP
EQIEYLESIVPFDVGFPKSLIGDDPAVTKKLSPLTSMSARIAFDN
Sequence ID 21 (corresponding to Sequence ID 8)
DNA Sequence
ATGACTGACTTGTTTAAACCTCTACCTGAACCACCTACCGAATTGGGACGTC
TCAGGGTTCTTTCTAAAACTGCCGGCATAAGGGTTTCACCGCTAATTCTGGG
AGGAGCTTCAATCGGCGACGCATGGTCAGGCTTTATGGGCTCTATGAATAA
GGAACAGGCCTTTGAACTTCTTGATGCTTTTTATGAAGCTGGAGGTAATTGT
ATTGATACTGCAAACAGTTACCAAAATGAAGAGTCAGAGATTTGGATAGGT
GAATGGATGGCATCAAGAAAACTGCGTGACCAGATTGTAATTGCCACCAAG
TTTACCGGAGA1TATAAGAAGTATGAAGTAGGTGGTGGTAAAAGTGCCAAC
TACTGTGGTAATCACAAGCGTAGTTTACATGTGAGTGTGAGGGATTCTCTCC
GCAAATTGCAAACTGATTGGATTGATATACTTTACATTCACTGGTGGGATTA
TATGAGTTCAATCGAAGAAGTTATGGATAGTTTGCATATTTTAGTTCAGCAG
GGCAAGGTCCTATATTTAGGAGTATCTGATACACCTGCTTGGGTTGTTTCTG
CGGCAAATTACTACGCTACATCTCATGGTAAAACTCCTTTTAGCGTCTATCA
AGGTAAATGGAATGTATTGAACAGGGACTTTGAGCGTGATATTATTCCAAT
GGCTAGGCATTTTGGTATGGCTCTAGCCCCATGGGATGTCATGGGAGGTGG
AAGATTTCAGAGTAAAAAAGCAATGGAAGAACGGAAGAAGAATGGAGAG
GGTCTGCGTACTTTTGTGGGTGGCCCCGAACAAACAGAATTGGAGGTTAAA
ATCAGCGAAGCATTGACTAAAATTGCTGAGGAACATGGAACAGAGTCTGTT
ACTGCTATCGCTATTGCCTATGTTCGCTCTAAAGCGAAAAATGTTTTCCCAT
TGATTGGAGGAAGGAAAATTGAACATCTCAAGCAGAACATTGAGGCTTTGA
GTATTAAATTAACACCGGAACAAATAGAATACCTGGAAAGTATTGTTCCTT
TTGATGTTGGCTTTCCCAAAAGTTTAATAGGAGATGACCCAGCGGTAACCA
AGAAGCTTTCACCCCTCACATCGATGTCTGCCAGGATAGCTTTTGACAATTA
G
Sequence Id No. 9
Amino acid Sequence
MCDSPATTGKPTILFIADPCETSATLNSKAFKEKFRILRYQLDTKEAFLNFLERH
EQDKICAIYAGFPAFKKIGGMTRSIIEHKSFPRKNLKCIVLCSRGYDGWDLDTLR
KHEIRLYNYQDDENEKLIDDLKLHQVGNDVADCALWHILEGFRKFSYYQKLSR
ETGNTLTARAKAAEKSGFAFGHELGNMFAESPRGKKCLILGLGSIGKQVAYKL
QYGLGMEIHYCKRSEDCTMSQNESWKFHLLDETIYAKLYQFHAIVVTLPGTHC
NPGLILVNLGRGKILDLRAVSDALVTGRINHLGLDVFNKEPEIDEKIRSSDRLTSI
TPHLGSATKDVFEQSCELALTRILRVVSGEAASDEHFSRVV
Sequence ID 22 (corresponding to Sequence ID 9)
DNA Sequence
ATGTGCGATTCTCCTGCAACGACTGGAAAGCCTACTATTCTTTTCATCGCAG
ATCCGTGCGAAACATCAGCCACACTTAATTCCAAGGCATTCAAAGAGAAGT
TCAGGATCTTGCGCTATCAGCTGGACACCAAAGAAGCATTTCTTAACTTTTT
AGAAAGGCATGAACAAGACAAAATATGTGCCATTTATGCTGGGTTTCCGGC
ATTCAAAAAAATCGGTGGGATGACTCGAAGTATCATCGAACACAAGTCATT
TCCAAGGAAAAATTTAAAATGTATCGTGCTTTGCTCAAGAGGTTACGACGG
ATGGGATCTGGATACATTACGCAAGCATGAAATTCGATTATACAACTACCA
AGACGATGAAAATGAAAAATTGATAGACGATTTAAAGCTTCATCAAGTCGG
TAATGATGTGGCAGATTGTGCCTTGTGGCACATTCTGGAGGGCTTTAGAAA
GTTCTCCTATTACCAAAAACTTAGTAGAGAAACTGGAAATACATTAACTGC
AAGGGCGAAAGCTGCAGAAAAGAGCGGATTTGCTTTTGGCCATGAACTGG
GGAATATGTTTGCTGAATCACCAAGAGGAAAGAAATGCTTAATTCTTGGTT
TAGGAAGTATTGGAAAGCAAGTAGCCTACAAGTTGCAATACGGGCTAGGA
ATGGAAATACATTATTGCAAAAGAAGCGAAGATTGCACAATGAGTCAAAA
CGAAAGCTGGAAATTTCATTTGCTAGATGAAACAATATATGCAAAACTATA
CCAGTTTCATGCAATCGTGGTCACATTGCCGGGAACTCCACAAACAGAACA
TTTAATCAACAGGAAATTTTTGGAACACTGCAATCCAGGCCTAATTTTAGTC
AACTTGGGAAGAGGTAAAATTTTGGACTTGCGGGCTGTTTCTGACGCCTTG
GTAACGGGACGAATCAACCATCTCGGTTTAGACGTCTTTAATAAAGAACCA
GAAATAGATGAAAAAATCAGATCTTCTGATAGACTTACTTCAATTACTCCG
CATTTGGGTAGTGCGACAAAGGATGTTTTTGAGCAAAGTTGTGAACTGGCA
TTGACAAGAATCTTACGGGTAGTGTCTGGGGAAGCCGCAAGCGATGAGCAT
TTCTCCCGTGTAGTTTGA
Sequence Id No. 10
Amino acid Sequence
MSSLVTLNNGLKMPLVGLGCWKIDKKVCANQIYEAIKLGYRLFDGACDYGNE
KEVGEGIRKAISEGLVSRKDIFVVSKLWNNFHHPDHVKLALKKTLSDMGLDYL
DLYYIHFPIAFKYVPFEEKYPPGFYTGADDEKKGHITEAHVPIIDTYRALEECVD
EGLIKSIGVSNFQGSLIQDLLRGCRIKPVALQIEHHPYLTQEHLVEFCKLHDIQV
VAYSSFGPQSFIEMDLQLAKTTPTLFENDVIKKVSQNHPGSTTSQVLLRWATER
LLGNLEIEKKFTLTEQELKDISALNANIRFNDPWTWLDGKFPTFA
Sequence ID 23 (corresponding to Sequence ID 10)
DNA Sequence
ATGTCTTCACTGGTTACTCTTAATAACGGTCTGAAAATGCCCCTAGTCGGCT
TAGGGTGCTGGAAAATTGACAAAAAAGTCTGTGCGAATCAAATTTATGAAG
CTATCAAATTAGGCTACCGTTTATTCGATGGTGCTTGCGACTACGGCAACGA
AAAGGAAGTTGGTGAAGGTATCAGGAAAGCCATCTCCGAAGGTCTTGTTTC
TAGAAAGGATATATTTGTTGTTTCAAAGTTATGGAACAATTTTCACCATCCT
GATCATGTAAAATTAGCTTTAAAGAAGACCTTAAGCGATATGGGACTTGAT
TATTTAGACCTGTATTATATTCACTTCCCAATCGCCTTCAAATATGTTCCATT
TGAAGAGAAATACCCTCCAGGATTCTATACGGGCGCAGATGACGAGAAGA
AAGGTCACATCACCGAAGCACATGTACCAATCATAGATACGTACCGGGCTC
TGGAAGAATGTGTTGATGAAGGCTTGATTAAGTCTATTGGTGTTTCCAACTT
TCAGGGAAGCTTGATTCAAGATTTATTACGTGGTTGTAGAATCAAGCCCGT
GGCTTTGCAAATTGAACACCATCCTTATTTGACTCAAGAACACCTAGTTGAG
TTTTGTAAATTACACGATATCCAAGTAGTTGCTTACTCCTCCTTCGGTCCTC
AATCATTCATTGAGATGGACTTACAGTTGGCAAAAACCACGCCAACTCTGT
TCGAGAATGATGTAATCAAGAAGGTCTCACAAAACCATCCAGGCAGTACCA
CTTCCCAAGTATTGCTTAGATGGGCAACTCAGAGAGGCATTGCCGTCATTC
CAAAATCTTCCAAGAAGGAAAGGTTACTTGGCAACCTAGAAATCGAAAAA
AAGTTCACTTTAACGGAGCAAGAATTGAAGGATATTTCTGCACTAAATGCC
AACATCAGATTTAATGATCCATGGACCTGGTTGGATGGTAAATTCCCCACTT
TTGCCTGA
Sequence Id No. 11
Amino acid Sequence
MANPTVIKLQDGNVMPQLGLGVWQASNEEVITAIQKALEVGYRSIDTAAAYK
NEEGVGKALKNASVNREELFITTKLWNDDHKRPREALLDSLKKLQLDYIDLYL
MHWPVPAIDHYVEAWKGMIELQKEGLIKSIGVCNFQIHHLQRLIDETGVTPVIN
QIELHPLMQQRQLHAWNATHKIQTESWSPLAQGGKGVFDQKVIRDLADKYGK
TPAQIVIRWHLDSGLVVIPKSVTPSRIAENFDVWDFRLDKDELGEIAKLDQGKR
LGPDPDQFGG
Sequence ID 24 (corresponding to Sequence ID 11)
DNA Sequence
ATGGCTAATCCAACCGTTATTAAGCTACAGGATGGCAATGTCATGCCCCAG
CTGGGACTGGGCGTCTGGCAAGCAAGTAATGAGGAAGTAATCACCGCCATT
CAAAAAGCGTTAGAAGTGGGTTATCGCTCGATTGATACCGCCGCGGCCTAC
AAGAACGAAGAAGGTGTCGGCAAAGCCCTGAAAAATGCCTCAGTCAACAG
AGAAGAACTGTTCATCACCACTAAGCTGTGGAACGACGACCACAAGCGCCC
CCGCGAAGCCCTGCTCGACAGCCTGAAAAAACTCCAGCTTGATTATATCGA
CCTCTACTTAATGCACTGGCCCGTTCCCGCTATCGACCATTATGTCGAAGCA
TGGAAAGGCATGATCGAATTGCAAAAAGAGGGATTAATCAAAAGCATCGG
CGTGTGCAACTTCCAGATCCATCACCTGCAACGCCTGATTGATGAAACTGG
CGTGACGCCTGTGATAAACCAGATCGAACTTCATCCGCTGATGCAACAACG
CCAGCTACACGCCTGGAACGCGACACACAAAATCCAGACCGAATCCTGGA
GCCCATTAGCGCAAGGAGGGAAAGGCGTTTTCGATCAGAAAGTCATTCGCG
ATCTGGCAGATAAATACGGCAAAACCCCGGCGCAGATTGTTATCCGCTGGC
ATCTGGATAGCGGCCTGGTGGTGATCCCGAAATCGGTCACACCTTCACGTA
TTGCCGAAAACTTTGATGTCTGGGATTTCCGTCTCGACAAAGACGAACTCG
GCGAAATTGCAAAACTCGATCAGGGCAAGCGTCTCGGTCCCGATCCTGACC
AGTTCGGCGGCTAA
Sequence Id No. 12
Amino acid Sequence
MAIPAFGLGTFRLKDDVVISSVITALELGYRAIDTAQIYDNEAAVGQAIAESGVP
RHELYITTKIWI
ENLSKDKLIPSLKESLQKLRTDYVDLTLIHWPSPNDEVSVEEFMQALLEAKKQG
LTREIGISNFTIPLMEKAIAAVGAENIATNQIELSPYLQNRKVVAWAKQHGIHIT
SYMTLAYGKALKDEVIARIAAKHNATPAQVILAWAMGEGYSVIPSSTKRKNLE
SNLKAQNLQLDAEDKKAIAALDCNDRLVSPEGLAPEWD
Sequence ID 25 (corresponding to Sequence ID 12)
DNA Sequence
ATGGCTATCCCTGCATTTGGTTTAGGTACTTTCCGTCTGAAAGACGACGTTG
TTATTTCATCTGTGATAACGGCGCTTGAACTTGGTTATCGCGCAATTGATAC
CGCACAAATCTATGATAACGAAGCCGCAGTAGGTCAGGCGATTGCAGAAA
GTGGCGTGCCACGTCATGAACTCTACATCACCACTAAAATCTGGATTGAAA
ATCTCAGCAAAGACAAATTGATCCCAAGTCTGAAAGAGAGCCTGCAAAAA
TTGCGTACCGATTATGTTGATCTGACGCTAATCCACTGGCCGTCACCAAACG
ATGAAGTCTCTGTTGAAGAGTTTATGCAGGCGCTGCTGGAAGCCAAAAAAC
AAGGGCTGACGCGTGAGATCGGTATTTCCAACTTCACGATCCCGTTGATGG
AAAAAGCGATTGCTGCTGTTGGTGCTGAAAACATCGCTACTAACCAGATTG
AACTCTCTCCTTATCTGCAAAACCGTAAAGTGGTTGCCTGGGCTAAACAGC
ACGGCATCCATATTACTTCCTATATGACGCTGGCGTATGGTAAGGCCCTGA
AAGATGAGGTTATTGCTCGTATCGCAGCTAAACACAATGCGACTCCGGCAC
AAGTGATTCTGGCGTGGGCTATGGGGGAAGGTTACTCAGTAATTCCTTCTTC
TACTAAACGTAAAAACCTGGAAAGTAATCTTAAGGCACAAAATTTACAGCT
TGATGCCGAAGATAAAAAAGCGATCGCCGCACTGGATTGCAACGACCGCCT
GGTTAGCCCGGAAGGTCTGGCTCCTGAATGGGATTAA
Sequence Id No. 13
Amino acid Sequence
MPATLHDSTKILSLNTGAQIPQIGLGTWQSKENDAYKAVLTALKDGYRHIDTA
AIYRNEDQVGQAIKDSGVPREEIFVTTKLWCTQHHEPEVALDQSLKRLGLDYV
DLYLMHWPARLDPAYIKNEDILSVPTKKDGSRAVDITNWNFIKTWELMQELPK
TGKTKAVGVSNFSINNLKDLLASQGNKLTPAANQVEIHPLLPQDELINFCKSKG
IVVEAYSPLGSTDAPLLKEPVILEIAKKNNVQPGHVVISWHVQRGYVVLPKSVN
STEDFEAINNISKEKGEKRVVHPNWSPFEVFK
Sequence ID 26 (corresponding to Sequence ID 13)
DNA Sequence
ATGCCTGCTACTTTACATGATTCTACGAAAATCCTTTCTCTAAATACTGGAG
CCCAAATCCCTCAAATAGGTTTAGGTACGTGGCAGTCGAAAGAGAACGATG
CTTATAAGGCTGTTTTAACCGCTTTGAAAGATGGCTACCGACACATTGATAC
TGCTGCTATTTACCGTAATGAAGACCAAGTCGGTCAAGCCATCAAGGATTC
AGGTGTTCCTCGGGAAGAAATCTTTGTTACTACAAAGTTATGGTGTACACA
ACACCACGAACCTGAAGTAGCGCTGGATCAATCACTAAAGAGGTTAGGATT
GGACTACGTAGACTTATATTTGATGCATTGGCCTGCCAGATTAGATCCAGCC
TACATCAAAAATGAAGACATCTTGAGTGTGCCAACAAAGAAGGATGGTTCT
CGTGCAGTGGATATCACCAATTGGAATTTCATCAAAACCTGGGAATTAATG
CAGGAACTACCAAAGACTGGTAAAACTAAGGCCGTTGGAGTCTCCAACTTT
TCTATAAATAACCTGAAAGATCTATTAGCATCTCAAGGTAATAAGCTTACG
CCAGCTGCTAACCAAGTCGAAATACATCCATTACTACCTCAAGACGAATTG
ATTAATTTTTGTAAAAGTAAAGGCATTGTGGTTGAAGCTTATTCTCCGTTAG
GTAGTACCGATGCTCCACTATTGAAGGAACCGGTTATCCTTGAAATTGCGA
AGAAAAATAACGTTCAACCCGGACACGTTGTTATTAGCTGGCACGTCCAAA
GAGGTTATGTTGTCTTGCCAAAATCTGTGAATCCCGATCGAATCAAAACGA
ACAGGAAAATATTTACTTTGTCTACTGAGGACTTTGAAGCTATCAATAACAT
ATCGAAGGAAAAGGGCGAAAAAAGGGTTGTACATCCAAATTGGTCTCCTTT
CGAAGTATTCAAGTAA

As used herein, the term “enzyme” refers to a polypeptide sequence encoded by a polynucleotide sequence which shows desirable enzymatic activity. The term ‘enzyme’ used anywhere in the specification would also include its suitable ‘variants’ as defined below, unless specified otherwise.

The term “variants” refers to polypeptides derived from the above nucleotide sequence by the addition, deletion, substitution or insertion of at least one nucleotide. As used herein, the terms “oxidoreductase,” or “oxidoreductase enzyme” refer to an enzyme that catalyzes the reduction of a ketone to form the corresponding alcohol in a stereoselective manner, optionally with the aid of co-factor.

As used herein, the term “co-factor” refers to an organic compound that operates in combination with an enzyme which catalyzes the reaction of interest. Co-factors include, for example, nicotinamide co-factors such as nicotinamide adenine dinucleotide (“NAD”), reduced nicotinamide adenine dinucleotide (“NADH”), nicotinamide adenine dinucleotide phosphate (“NADP⁺”), reduced nicotinamide adenine dinucleotide phosphate (“NADPH”), and any derivatives or analogs thereof.

The term “expression construct” as used herein comprises a nucleotide sequence of interest to express and control the expression of gene/s of interest.

The term as used herein “monocistronic expression construct” means that the expression construct is expressing a single gene.

The term as used herein “polycistronic expression construct” means that two or more genes are being expressed in a single expression construct.

The term as used herein “enzyme coupled co-factor regeneration system” means the expression of a suitable enzymatic polypeptide in an expression vector having the potential to regenerate reduced cofactor from oxidized NAD(P) during the reaction.

The term as used herein “substrate coupled co-factor regeneration system” means the use of a suitable substrate H⁺ donor having potential to regenerate reduced cofactor from oxidized NAD(P) during the reaction.

pET11aZBG5.1.1 is an expression vector that encodes a gene sequence of Sequence Id No. 1 which is representing the Genbank Id no. NP_{—}579689.1 under the control of a T7 promoter in the vector pET11a utilizing ampicillin drug resistance marker.

pET11aZBG6.4.1 is an expression vector that encodes a gene sequence of Sequence Id No. 2 which is representing the Genbank Id no YP_{—}399703.1 under the control of a T7 promoter in the vector pET11a utilizing ampicillin drug resistance marker.

pET11aZBG2.0.1 is an expression vector that encodes a gene sequence of Sequence Id No. 3 which is representing the Genbank Id no NP_{—}013953.1 under the control of a T7 promoter in the vector pET11a utilizing ampicillin drug resistance marker.

pET11aZBG25.1.1 is an expression vector that encodes a gene sequence of Sequence Id No. 4 which is representing the Genbank Id no AAA21973.1 under the control of a T7 promoter in the vector pET11a utilizing ampicillin drug resistance marker.

pET11aZBG8.1.1 is an expression vector that encodes a gene sequence of Sequence Id No. 5 which is representing the Genbank Id no BAH28833.1 under the control of a T7 promoter in the vector pET11a utilizing ampicillin drug, resistance marker.

pET11aZBG13.1.1 is an expression vector that encodes a gene sequence of Sequence Id No. 7 which is representing the Genbank Id no AAX31145.1 under the control of a T7 promoter in the vector pET11a utilizing ampicillin drug resistance marker.

pET27bZBG5.1.1 is an expression vector that encodes a gene sequence of Sequence Id No. 1 which is representing the Genbank Id no. NP_{—}579689.1 under the control of a T7 promoter in the vector pET27b utilizing kanamycin drug resistance marker.

pET27bZBG2.0.1 is an expression vector that encodes a gene sequence of Sequence Id No. 3 which is representing the Genbank Id no. NP_{—}013953.1 under the control of a T7 promoter in the vector pET27b utilizing kanamycin drug resistance marker.

pET27bZBG8.1.1 is an expression vector that encodes a gene sequence of Sequence Id No. 5 which is representing the Genbank Id no. BAH28833.1 under the control of a T7 promoter in the vector pET27b utilizing kanamycin drug resistance marker.

pET27bZBG2.0.9 is an expression vector that encodes a gene sequence of Sequence Id No. 6 which is representing the Genbank Id no. NP_{—}012630.1 under the control of a T7 promoter in the vector pET27b utilizing kanamycin drug resistance marker.

pET27bZBG13.1.1 is an expression vector that encodes a gene sequence of Sequence Id No. 7 which is representing the Genbank Id no. AAX31145.1 under the control of a T7 promoter in the vector pET27b utilizing kanamycin drug resistance marker.

pET27bZBG2.0.8 is an expression vector that encodes a gene sequence of Sequence Id No. 8 which is representing the Genbank Id no. NP_{—}014068 under the control of a T7 promoter in the vector pET27b utilizing kanamycin drug resistance marker

pET27bZBG2.0.11 is an expression vector that encodes a gene sequence of Sequence Id No. 9 which is representing the Genbank Id no. NP_{—}011330 under the control of a T7 promoter in the vector pET27b utilizing kanamycin drug resistance marker

pET27bZBG2.0.5 is an expression vector that encodes a gene sequence of Sequence Id No. 10 which is representing the Genbank Id no. NP_{—}011972.1 under the control of a T7 promoter in the vector pET27b utilizing kanamycin drug resistance marker.

pET27bZBG1.1.22 is an expression vector that encodes a gene sequence of Sequence Id No. 11 which is representing the Genbank Id no. ACB04098.1 under the control of a T7 promoter in the vector pET27b utilizing kanamycin drug resistance marker.

pET27bZBG1.1.2 is an expression vector that encodes a gene sequence of Sequence Id No. 12 which is representing the Genbank Id no. ACB01380.1 under the control of a T7 promoter in the vector pET27b utilizing kanamycin drug resistance marker.

pET27bZBG2.0.4 is an expression vector that encodes a gene sequence of Sequence Id No. 13 which is representing the Genbank Id no. NP_{—}014763.1 under the control of a T7 promoter in the vector pET27b utilizing kanamycin drug resistance marker.

The term used herein “whole cell” means a recombinant E. coli deposited under Budapest treaty, having accession number MTCC 5642, MTCC 5643, MTCC 5644, MTCC 5645, MTCC 5646, MTCC 5647, MTCC 5648, MTCC 5649, MTCC 5650, MTCC 5651, MTCC 5652, MTCC 5653, MTCC 5654.

The term “Metal ion salt” refers to Na, K, Li, Ca, Mg, Cu and Cs.

The present invention provides a process for the preparation of suitable intermediate of formula (I)

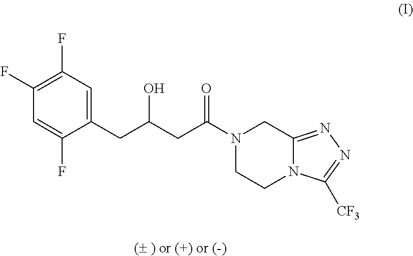 [SMILES: O=C(CC(O)Cc1cc(F)c(F)cc1F)N1CCn2c(nnc2C(F)(F)F)C1]

comprising:
c) reacting 4-oxo-4-[3-(trifluoromethyl)-5,6-dihydro[1,2,4]triazolo[4,3-a]pyrazin-7(8H)-yl]-1-(2,4,5-trifluorophenyl)butan-2-one of formula (III) or its metal ion salt:

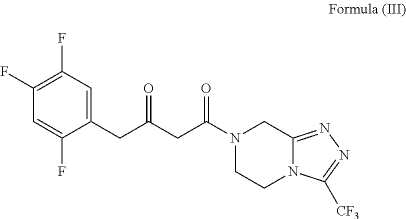 [SMILES: O=C(CC(=O)N1CCn2c(nnc2C(F)(F)F)C1)Cc1cc(F)c(F)cc1F]

with a suitable enzyme that stereoselectively reduces a ketone to form an alcohol, by maintaining under suitable conditions and cofactor
d) isolating the suitable intermediate.

The invention provides two enantiomers of 3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one of opposite chirality of the following formulae:

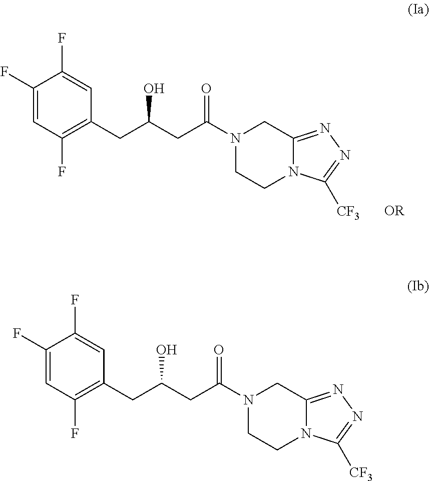 [SMILES: O=C(C[C@H](O)Cc1cc(F)c(F)cc1F)N1CCn2c(nnc2C(F)(F)F)C1 ; O=C(C[C@@H](O)Cc1cc(F)c(F)cc1F)N1CCn2c(nnc2C(F)(F)F)C1]

The invention is directed to processes for the preparation of 3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one [Formula (I)], either in racemic (R/S) form or any of its optically active (R) or (S) forms [Formula (Ia) and (Ib) respectively], via enzymatic reduction of 4-oxo-4-[3-(trifluoromethyl)-5,6-dihydro[1,2,4]triazolo[4,3-a]pyrazin-7(8H)-yl]-1-(2,4,5-trifluorophenyl)butan-2-one of formula (III) or its metal ion salt: comprising;
a) a) reacting 4-oxo-4-[3-(trifluoromethyl)-5,6-dihydro[1,2,4]triazolo[4,3-a]pyrazin-7(8H)-yl]-1-(2,4,5-trifluorophenyl)butan-2-one of formula (III) or its metal ion salt:

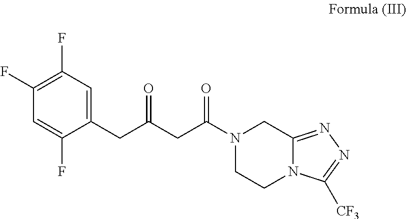 [SMILES: O=C(CC(=O)N1CCn2c(nnc2C(F)(F)F)C1)Cc1cc(F)c(F)cc1F]

with a suitable enzyme and its variants that stereoselectively reduce a ketone to form an alcohol, by maintaining under suitable conditions, to obtain 3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one, in racemic (R/S) form or any of its optically active (S) or (R) forms or their enantiomerically excess mixtures.

In one embodiment of the present invention the polypeptide having desired enzymatic activity and variants thereof can be isolated from suitable bacteria, yeast or fungi. In one embodiment the suitable polypeptides having enzymatic activities are selected from oxidoreductases. In a preferred embodiment suitable enzymes are to selected from aldo-keto reductases. In an another embodiment suitable enzymes are selected from dehydrogenases. In an embodiment the NAD(P)⁺ dependent reductase is selected from the Saccharomyces species. In an another preferred embodiment NAD(P)⁺ dependent reductase is selected (derived) from Saccharomyces cerevisiae and having Genebank id:—NP_{—}012630.1. In an another preferred embodiment NAD(P)⁺ dependent alcohol dehydrogenase is selected (derived) from Saccharomyces cerevisiae and having Genebank id:—NP_{—}013953.1, NP_{—}014763.1, NP_{—}011972.1, NP_{—}014068 and NP_{—}011330.

In a preferred embodiment suitable enzymes are selected from short chain dehydrogenases. Examples of such short chain dehydrogenases include NAD(P)⁺/NAD(P)H⁺ dependent alcohol dehydrogenases In another embodiment the short chain dehydrogenase is selected from NAD(P)H dependent 3-quinuclidinone reductase. In an embodiment NAD(P)H dependent 3-quinuclidinone reductase is selected from Rhodotorula species. In a preferred embodiment NAD(P)H dependent-3-quinuclidinone reductase is selected from Rhodotorula mucilaginosa and having Genebank id:—BAH28833.1.

In another embodiment the enzymes are selected from suitable aldoketo reductases. Examples of such aldoketo-reductase include aldose-reductase, aldehyde reductase, carbonyl reductse and ketoreductase. In an embodiment the ketoreductase is selected from Pichia species. In a preferred embodiment NAD(P)⁺ dependent ketoreductase is selected from Pichia methanolica and having Genebank id:—AAW06921.1.

In another embodiment the aldose reductase is selected from Pyrococcus species. In such embodiment aldose reductase is selected from Pyrococcus furiosus and having Genebank id:—NP_{—}579689.1.

In another embodiment the acetoacetyl reductase is selected from Cupriavidus species. In such embodiment aldose reductase is selected from Cupriavidus necator and having Genebank id:—AAA21973.1

In another preferred embodiment aldose reductase preferably 2,5-diketo-D-gluconate reductase B is selected from Escherichia coli and having Genebank id:—YP_{—}002998068.1.

In another preferred embodiment aldose reductase preferably 2,5-diketo-D-gluconate reductase A is selected from Escherichia coli and having Genebank id:—ACB04098.1

In embodiment the genes which encode polypeptides or their variants of desired enzymatic activity are cloned into suitable vectors which can be selected from plasmid vector, a phage vector, a cosmid vector and shuttle vector may be used that can exchange a gene between host strains. Such vectors typically include a control element, such as a lacUV5 promoter, a trp promoter, a trc promoter, a tac promoter, a lpp promoter, a tufB promoter, a recA promoter, or a pL promoter, and are preferably employed as an expression vector including an expression unit operatively linked to the polynucleotide of the present invention.

The genes which encode polypeptides or their variants of desired enzymatic activity are selected from sequences which are set forth in Sequence Id No. 1, Sequence Id No. 2, Sequence Id No. 3, Sequence Id No. 4, Sequence Id No. 5, Sequence Id No. 6, Sequence Id No. 8, Sequence Id No. 9, Sequence Id No. 10, Sequence Id No. 11, Sequence Id No. 12 and Sequence Id No. 13 or their variants. In a preferred embodiment the polynucleotide of sequences encoding these polypeptides having oxidoreductase enzymatic activity are cloned in a cloning vector construct pET11a or pET27b, according to general techniques described in Sambrook et al, Molecular cloning, Cold Spring Harbor Laboratories (2001). The constructed vectors are now onwards referred to as pET11aZBG5.1.1, pET11aZBG6.4.1, pET11aZBG2.0.1, pET11aZBG25.1.1, pET11aZBG8.1.1, pET11aZBG13.1.1, pET27bZBG5.1.1, pET27bZBG2.0.1, pET27bZBG8.1.1, pET27bZBG2.0.9, pET27bZBG13.1.1, pET27bZBG2.0.8, pET27bZBG2.0.11, pET27bZBG2.0.5, pET27bZBG1.1.22, pET27bZBG1.1.2, and pET27bZBG2.0.4.

In addition, these vectors further contain a gene encoding an enzyme which can regenerate the co-factors such as NAD, NADP, NADH, NADPH.

The term “control element” as used herein refers to a functional promoter and a nucleotide sequence having any associated transcription element (e.g., enhancer, CCAAT box, TATA box, SPI site).

The polynucleotide of the present invention is linked with control elements, such as a promoter and an enhancer, which control the expression of the gene in such a manner that the control elements can operate to express and regulate the expression of the gene. It is well known to those skilled in the art that the types of control elements may vary depending on the host cell.

In an embodiment the present process provides a vector construct comprising monocistronic expression construct of nucleotide sequence encoding the polypeptide having desired oxidoreductase enzymatic activity. Alternatively the vector construct comprising monocistronic expression construct of nucleotide sequence is encoding the polypeptide having the potential to generate co-factor from oxidized NAD(P) during the reaction.

According to such embodiment the oxidoreductase polypeptide encoded by nucleotide sequence is selected from Sequence Id No. 1, Sequence Id No. 2, Sequence Id No. 3, Sequence Id No. 4, Sequence Id No. 5, Sequence Id No. 6, Sequence Id No. 8, Sequence Id No. 9, Sequence Id No. 10, Sequence Id No. 11, Sequence Id No. 12 and Sequence Id No. 13 or its variants, and is coupled with the cofactor selected from NAD(P)H/NAD(P) to produce the optically pure formula-(I), or in racemic (R/S) form or any of its optically active (S) or (R) forms or their enantiomerically excess mixtures by reduction of the compound of formula-(III) wherein the cofactor is either added externally in reaction medium or obtained by enzyme/substrate coupled regeneration system.

In an embodiment the present process provides a vector construct comprising polycistronic expression construct of nucleotide sequences encoding the polypeptide having desired oxidoreductase enzymatic activity and the polypeptide having potential to generate co-factor from oxidized NAD(P) during the reaction.

According to such embodiment the oxidoreductase polypeptide of sequence IDs selected from sequence id1 to sequence id 13 (except sequence id7) which is disclosed in present invention is coupled with the cofactor selected from NAD(P)H/NAD(P) to produce 3,3-hydroxy-1-(3-(tri fluoromethyl)-5,6-dihydro-[1,2,4]-triazolo-[4,3-a]-pyrazin-7(8H)-yl)-4-(2,4,5-trifluorophenyl)butan-1-one, in its racemic (R/S) form or any of its optically active (S) or (R) forms or enantiomeric excess mixture of any of the forms by reduction of 4-oxo-4-[3-(trifluoromethyl)-5,6-dihydro[1,2,4]triazolo[4,3-a]pyrazin-7(8H)-yl]-1-(2,4,5-trifluorophenyl)butan-2-one of formula (III) wherein the cofactor regenerating enzyme is co expressed with nucleotide sequence encoding polypeptide having oxidoreductase activity in the same vector.

In an embodiment the vector is having potential to co-express oxidoreductase polypeptide of sequence selected from Sequence Id No. 1, Sequence Id No. 2, Sequence Id No. 3, Sequence Id No. 4, Sequence Id No. 5, Sequence Id No. 6, Sequence Id No. 8, Sequence Id No. 9, Sequence Id No. 10, Sequence Id No. 11, Sequence Id No. 12 and Sequence Id No. 13 or its variants disclosed in present invention along with polypeptide having potential to generate co-factor from oxidized NAD(P) during the reaction comprising;
a. at least one region that controls the replication and maintenance of said vector in the host cell;
b. first promoter operably linked to the nucleotide sequence encoding the amino acid sequences setforth in Sequence Id No. 1, Sequence Id No. 2, Sequence Id No. 3, Sequence Id No. 4, Sequence Id No. 5, Sequence Id No. 6, Sequence Id No. 8, Sequence Id No. 9, Sequence Id No. 10, Sequence Id No. 11, Sequence Id No. 12 and Sequence Id No. 13 or their variants, disclosed in the present invention, encoding the oxidoreductase enzyme;
c. second promoter operably linked to the nucleotide sequence encoding the a.a. sequence setforth in sequence ID no 7 or variant thereof encoding polypeptide having potential to regenerate co-factor;
d. suitable antibiotic marker

In an embodiment the gene positions are changeable and therefore position of sequence IDs mentioned in steps (b) and (c) of above described vector are replaceable with each other.

In an embodiment vectors are selected from pET11aZBG5.1.1, pET11aZBG6.4.1, pET11aZBG2.0.1, pET11aZBG25.1.1, pET11aZBG8.1.1, pET11aZBG13.1.1, pET27bZBG5.1.1, pET27bZBG2.0.1, pET27bZBG8.1.1, pET27bZBG2.0.9, pET27bZBG13.1.1, pET27bZBG2.0.8, pET27bZBG2.0.11, pET27bZBG2.0.5, pET27bZBG1.1.22, pET27bZBG1.1.2, pET27bZBG2.0.4

According to the present invention monocistronic or polycistronic vectors containing polynucleotides or their variants having desired oxidoreductase enzymatic activity are transfected in to the host cells using a calcium chloride method as known in the art. The host cell may be selected from bacteria, yeast, molds, plant cells, and animal cells. In a preferred embodiment the host cell is a bacteria such as Escherichia coli. In such embodiment the above mentioned desired polypeptides are over-expressed in E. coli.

According to preferred embodiment the invention provides a process for the production of the compound of formula (I), or any of its optically active (S) or (R) forms or enantiomeric excess mixture of any of the forms which comprise the steps:
a) dissolution of the compound of formula (III) or its metal ion salt in suitable solvent;
b) reacting the compound of formula (III) or its metal ion salt with suitable oxidoreductase enzyme in the presence of suitable conditions and cofactor;
c) optionally maintain the pH during the reaction;
d) isolating the compound of formula (I), or any of its optically active (S) or (R) forms or enantiomeric excess mixture of any of the forms.

The oxidoreductase enzymes suitable for the reaction share at least 50% homology/identity with the sequence IDs disclosed in the present invention or its variants.

In one such embodiment the cofactor is added externally in reaction medium. In an alternate embodiment the co factor is obtained by enzyme coupled regeneration system. The enzyme which is used in enzyme coupled regeneration system is selected from glucose dehydrogenase, formate dehydrogenase, malate dehydrogenase, glucose-6-phosphate dehydrogenase, phosphite dehydrogenase. In one preferred embodiment the enzyme is glucose dehydrogenase. In one such embodiment oxidoreductase enzyme is expressed in monocistronic vector. In another embodiment oxidoreductase enzyme is co-expressed with glucose dehydrogenase in a polycistronic vector in a single expression system. In such a preferred embodiment, the expression system is bacteria, such as Escherichia coli.

In another embodiment, oxidoreductase polypeptide (encoded by nucleotide sequence) selected from which is set forth in Sequence Id No. 1, Sequence Id No. 2, Sequence Id No. 3, Sequence Id No. 4, Sequence Id No. 5, Sequence Id No. 6, Sequence Id No. 8, Sequence Id No. 9, Sequence Id No. 10, Sequence Id No. 11, Sequence Id No. 12 and Sequence Id No. 13 or its variants disclosed in the present invention, is coupled with the cofactor selected from NAD(P)H/NAD(P) to produce the optically compound of formula (I), or any of its optically active (S) or (R) forms or enantiomeric excess mixture of any of the forms by reduction of the formula-(III) wherein the cofactor is regenerated through substrate coupled regeneration system.

The substrate coupled regeneration system comprises co-substrate selected from ethanol, 2-propanol, 4-methyl-2-pentanol, 2-heptanol, 2-pentanol, 2-hexanol. In preferred embodiment the co-substrate used in substrate coupled regeneration system is 2-propanol.

Moreover, the substrate coupled regeneration system requires the action of at least one enzyme. In preferred embodiment the substrate coupled regeneration system requires the action of enzyme comprising the polypeptide as set forth in sequence IDs to disclosed in the invention or variants thereof. According to preferred embodiment of the process sequence IDs disclosed in the present invention or variants are expressed in monocistronic vector.

According to preferred embodiment the reduced co-factor such as NAD(P)H is regenerated by dehydrogenation of the 2-propanol by the enzyme of IDs disclosed in the present invention or variants to produce acetone. Furthermore the reduced co-factor couples with the said enzyme and reacts with substrate according to acid-base catalytic mechanism. Thus, in this process the reduced co-factor NAD(P)H is regenerated continuously by dehydrogenation of alcohol by the same oxidoreductase enzyme.

In one embodiment the optically pure compound of formula (I), or any of its optically active (S) or (R) forms or enantiomeric excess mixture of any of the forms is produced by reduction of the formula-(III) in suitable reaction condition with the cell-free extracts which comprises the desired sequence selected from which is set forth in Sequence Id No. 1, Sequence Id No. 2, Sequence Id No. 3, Sequence Id No. 4, Sequence Id No. 5, Sequence Id No. 6, Sequence Id No. 8, Sequence Id No. 9, Sequence Id No. 10, Sequence Id No. 11, Sequence Id No. 12 and Sequence Id No. 13 or its variants disclosed in the present invention. The cell free extract is obtained from the lysis of the host cell comprising the monocistronic vector containing the polynucleotide sequence encoding the oxidoreductase enzyme and its variants according to sequence selected from which is set forth in Sequence Id No. 1, Sequence Id No. 2, Sequence Id No. 3, Sequence Id No. 4, Sequence Id No. 5, Sequence Id No. 6, Sequence Id No. 8, Sequence Id No. 9, Sequence Id No. 10, Sequence Id No. 11, Sequence Id No. 12 and Sequence Id No. 13 or its variants disclosed in the present invention and the required cofactor may be added externally. Alternatively, the cell free extract is obtained from the lysis of the host cell comprising the polycistronic vector containing the polynucleotide sequence encoding the oxidoreductase enzyme and its variants according to IDs disclosed in the present invention and polypeptide in vector having potential to regenerate cofactor from oxidized NAD(P).

Optionally the cell free extract may be lyophilized or dried to remove water by the processes known in the art such as lyophilization or spray drying. The dry powder obtained from such processes comprises at least one oxidoreductase enzyme and its variants according to sequence IDs disclosed in the present invention which may be used to form optically pure formula (I), or any of its optically active (S) or (R) forms or enantiomeric excess mixture of any of the forms by reduction of the formula-(III) or to its metal ion salt.

In an embodiment the optically pure formula (I), or any of its optically active (S) or (R) forms or enantiomeric excess mixture of any of the forms by reduction of the formula-(III) in suitable reaction condition with the whole cells biocatalyst which comprises at least the desired polypeptide or its variants encoded by nucleotide sequence selected from which is set forth in Sequence Id No. 1 and 14, Sequence Id No. 2 and 15, Sequence Id No. 3 and 16, Sequence Id No. 4 and 17, Sequence Id No. 5 and 18, Sequence Id No. 6 and 19, Sequence Id No. 8 and 21, Sequence Id No. 9 and 22, Sequence Id No. 10 and 23, Sequence Id No. 11 and 24, Sequence Id No. 12 and 25 and Sequence Id No. 13 and 26 or its variants and the cofactor may be added externally during the reaction.

According to the preferred embodiment the invention provides a process for the production of the compound of formula (I), or any of its optically active (S) or (R) forms or enantiomeric excess mixture of any of the forms which comprises
a) dissolving the compound of formula (III) or its metal ion salt in suitable solvent
b) reacting the compound of formula (III) or its metal ion salt with suitable recombinant whole cell which comprises an expression vector which co-expresses the oxidoreductase enzyme and polypeptide having potential to regenerate co-factor, wherein the oxidoreductase enzyme is selected from sequence IDs of the present invention and its variants.
c) maintaining the pH during the reaction
d) isolating of the compound of formula (I), or any of its optically active (S) or (R) forms or enantiomeric excess mixture of any of the forms

In such embodiment the whole cell is selected from recombinant E. coli having accession number MTCC 5642, MTCC 5643, MTCC 5644, MTCC 5645, MTCC 5646, MTCC 5647, MTCC 5648, MTCC 5649, MTCC 5650, MTCC 5651, MTCC 5652, MTCC 5653, MTCC 5654 which expresses the desired polypeptide sequences as set forth in sequence IDs disclosed in the present invention or their variants and polypeptide having capacity to regenerates the reduced form of NAD(P)H.

In yet another embodiment the optically pure formula (I), or any of its optically active (S) or (R) forms or enantiomeric excess mixture is prepared by reacting the formula (III) or its metal ion salt in suitable reaction condition with the isolated and purified desired polypeptide as shown in sequence IDs disclosed in the present invention or its variants which shows at least 50% homology with the sequence IDs of the present invention.

In one general embodiment of the process according to the invention, the ketone of formula (III) is preferably used in an amount of from 0.1 to 30% W/V. In a preferred embodiment, the amount of ketone is 10% W/V. The process according to the invention is carried out in aqueous system. In such embodiment the aqueous portion of the reaction mixture in which the enzymatic reduction proceeds preferably contains a buffer. Such buffer is taken in the range of 50-200 mM is selected from sodium succinate, sodium citrate, phosphate buffer, Tris buffer. The pH is maintained from about 5 to 9 and the reaction temperature is maintained from about 15° C. to 50° C. In a preferred embodiment the pH value is 7 to 8 and the temperature ranges from 25° C. to 40° C.

Alternatively, the reaction can be carried out in an aqueous solvent in combination with organic solvents. Such aqueous solvents include buffers having buffer capacity at a neutral pH, are selected from phosphate buffer and Tris-HCl buffer. Alternatively, no buffer is required when the use of acid and alkali can keep the pH change during the reaction within a desired range Organic solvents are selected from n-butanol, Iso propyl alcohol, ethyl acetate, butyl acetate, toluene, chloroform, n-hexane, ethanol, acetone, dimethyl sulfoxide, and acetonitrile etc. In another embodiment, the reaction is performed without buffer in presence of acid and alkali which maintain the pH change during the reaction within a desired range. Alternatively, the reaction can be carried out in a mixed solvent system consisting of water miscible solvents such as ethanol, acetone, dimethyl sulfoxide, and acetonitrile.

The Polypeptide having desired enzymatic activity encoded by the nucleotide sequence selected from those set forth in Sequence Id No. 1, Sequence Id No. 2, Sequence Id No. 3, Sequence Id No. 4, Sequence Id No. 5, Sequence Id No. 6, Sequence Id No. 8, Sequence Id No. 9, Sequence Id No. 10, Sequence Id No. 11, Sequence Id No. 12 and Sequence Id No. 13 or its variants disclosed in the present invention or its variants thereof is used in concentration of at least 5 mg/mL of lyophilized and water-resuspended crude lysate.

Furthermore, in such embodiment, optionally the NAD(P) formed with the enzymatic reduction of NAD(P)H can again be converted to NAD(P)H with the oxidation of co substrate selected from Ethanol, 2-propanol, 4-methyl-2-pentanol, 2-heptanol, 2-pentanol, 2-hexanol. Moreover, the concentration of the cofactor NAD(P) or NAD(P)H respectively is selected from 0.001 mM to 100 mM.

In one preferred embodiment the reduction of the formula (III) or its metal ion salt is carried out by the same polypeptide encoded by polynucleotide of sequence IDs disclosed in the present invention or its variants.

In another embodiment the reduction of the formula (III) or its metal ion salt is carried out by the nucleotide sequences selected from which is set forth in Sequence Id No. 1, Sequence Id No. 2, Sequence Id No. 3, Sequence Id No. 4, Sequence Id No. 5, Sequence Id No. 6, Sequence Id No. 8, Sequence Id No. 9, Sequence Id No. 10, Sequence Id No. 11, Sequence Id No. 12 and Sequence Id No. 13 or their variants in combination with the polypeptides selected from Glucose dehydrogenase, Formate dehydrogenase, Malate dehydrogenase, Glucose-6-Phosphate dehydrogenase, Phosphite dehydrogenase.

In such embodiment, the cofactor is regenerated by the oxidation of glucose used as co-substrate in the presence of Glucose dehydrogenase in suitable concentration such that its concentration is at least 0.1-10 times higher molar concentration than the keto substrate. In such embodiment the enzyme concentration is selected from at least 5 mg/mL of lyophilized and water-resuspended crude lysate.

According to the present invention, a process for the preparation of formula (I), or any of its optically active (S) or (R) forms or enantiomeric excess mixture of any of the forms can be carried out by various processes including the use of recombinant host cell, cell free extract/crude lysate obtained from recombinant host cell, isolated desired enzyme which is isolated from cell free extract/crude lysate or from the suitable organism.

At the end of the reaction when the product are formed, thereafter the product is isolated from the reaction mixture from techniques known in the art.

The (S) or (R)-3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one or their enantiomerically excess mixtures, obtained as above, are suitable as intermediate for the preparation of Sitagliptin.
(S)-3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one can be converted to (S)-3-(methanesulfonyloxy)-1-(3-(trifluoromethyl)-5,6-dihydro-[1,2,4]triazolo[4,3-a]pyrazin-7(8H)-yl)-4-(2,4,5-trifluorophenyl)butan-1-one by reacting with methanesulfonyl chloride; which can be further converted to ((R)-3-azido-1-[3-(trifluoromethyl)-5,6-dihydro[1,2,4]triazolo[4,3-a]pyrazin-7(8H)-yl]-4-(2,4,5-trifluorophenyl)butan-1-one by reacting with sodium azide which can be further converted to (R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine (Sitagliptin) by using Pd/c and sodium borohydride. Similarly, (S)-4-oxo-4-[3-(trifluoromethyl)-5,6-dihydro[1,2,4]triazolo[4,3-a]pyrazin-7(8H)-yl]-(2,4,5-trifluorophenyl)butan-2-amine can be obtained from (R)-3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one.

In another aspect there is provided a novel intermediate of Formula (IVa) optically active (S) and Formula (IVb) optically active (R) forms or their enantiomerically excess mixtures which can be used in the preparation of the compound of Formula (II).

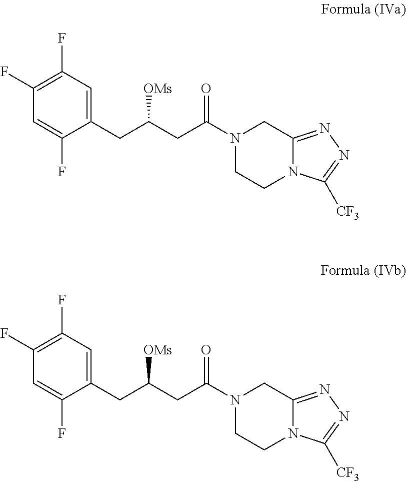 [SMILES: CS(=O)(=O)O[C@H](CC(=O)N1CCn2c(nnc2C(F)(F)F)C1)Cc1cc(F)c(F)cc1F ; CS(=O)(=O)O[C@@H](CC(=O)N1CCn2c(nnc2C(F)(F)F)C1)Cc1cc(F)c(F)cc1F]

In another aspect there is provided a novel intermediate of Formula (Va) optically active (S) and Formula (Vb) optically active (R) forms or their enantiomerically excess mixtures which can be used in the preparation of the compound of Formula (II).

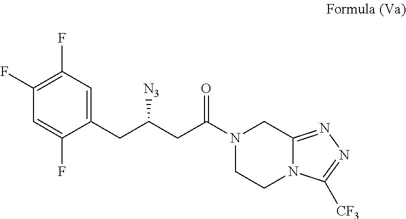 [SMILES: [N-]=[N+]=N[C@H](CC(=O)N1CCn2c(nnc2C(F)(F)F)C1)Cc1cc(F)c(F)cc1F]

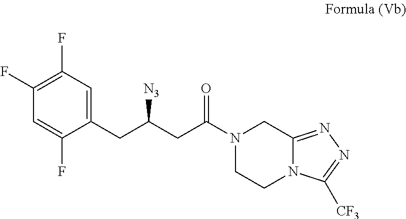 [SMILES: [N-]=[N+]=N[C@@H](CC(=O)N1CCn2c(nnc2C(F)(F)F)C1)Cc1cc(F)c(F)cc1F]

The present invention is further exemplified which are provided for the illustration purpose but the scope of the present invention is not limited with the only below given examples.

### Example 1

### Cloning and Gene Expression Analysis of Chemically Synthesized Oxidoreductase and Co-Factor Regenerating Enzymes

DNA, sequences deduced from the polypeptide sequences shown in sequence id nos. 1, 2, 3, 4, 5 and 7 were codon optimized for expression in E. coli and were cloned in a pET11a plasmid vector. In each case, the ligated DNA was further transformed into competent E. coli cells and the transformation mix was plated on Luria agar plates containing ampicillin. The positive clones were identified on the basis of their utilizing ampicillin resistance for growth on the above petri plates and further restriction digestion of the plasmid DNA derived from them. Clones giving desired fragment lengths of digested plasmid DNA samples were selected as putative positive clones. With each DNA sequence, one of such putative positive clones was submitted to nucleotide sequence analysis and was found to be having 100% homology with the sequence used for chemical synthesis. These pET11a clones corresponding to sequence Id nos. 1, 2, 3, 4, 5 and 7 were named respectively as per Table no. 1A. Plasmid DNA isolated from these clones were transformed into the E. coli expression host, BL21 (DE3), and plated on ampicillin containing Luria Agar plates followed by overnight incubation at 37° C. Colonies for each clone were picked from the respective plates and grown in Luria Broth containing ampicillin and the plasmid DNA isolated from the respective cultures were further subjected to restriction digestion analysis using the respective restriction enzymes to confirm the correctness of the clone. Also these cultures were subjected to induction with suitable concentration (0.01-2 mM) of IPTG for expression analysis. Simultaneously IPTG induced cultures were lysed and clarified lysates obtained after centrifugation were subjected to SDS-PAGE analysis to confirm induced expression of polypeptide of correct size. After confirming the restriction fragment analysis and expression analysis, the fresh culture of these clones were used for the preparation of glycerol stocks. These clones were used as a source of enzymatic polypeptide of Seq ID nos. 1, 2, 3, 4, 5 and 7 for subsequent biocatalysis studies.

To make the process more environmental friendly, a better class of antibiotic was chosen and subcloning of some of the above enzymes was done in pET27 b (+), a vector having a kanamycin resistance gene instead of ampicillin. All other components of the vector were similar to pET11a. Briefly, the plasmid DNA from pET11a clones were digested with the cloning enzymes NdeI-BamHI to excise the gene from the vector. After digestion with these enzymes the DNA corresponding to sequence Id nos. to 1, 3, 5 and 7 as shown in table no. 1 were ligated with pET27b(+) plasmid vector pre-digested with the cloning enzymes NdeI-BamHI. The ligated DNA was further transformed into competent E. coli Top10F′ cells and the transformation mix was plated on Luria agar plates containing kanamycin. The positive clones were identified on the basis of their utilizing kanamycin resistance for growth on the above petri plates and is further restriction digestion of the plasmid DNA derived from them with the respective internally cutting enzymes for both vector and insert. One such clone giving desired fragment lengths of digested plasmid DNA samples was selected as a putative positive clone. One of the putative positive clones of pET27b was selected and named as per table no. 1A. Plasmid DNA isolated from these pET27b clones were transformed into the E. coli expression host, BL21(DE3), and plated on kanamycin containing Luria Agar plates followed by incubation at 37° C. for overnight. Colonies picked from this plate were grown in Luria Broth containing kanamycin, and the plasmid DNA isolated from these cultures were further subjected to restriction digestion analysis using the respective restriction enzymes to confirm the correctness of the clone. Also these cultures were subjected to induction with suitable concentration (0.01-2 mM) of IPTG for expression analysis. Simultaneously, IPTG induced cultures were lysed and clarified lysates obtained after centrifugation were subjected to SDS-PAGE analysis to confirm induced expression of polypeptide of correct size. After confirming the restriction fragment analysis and expression analysis, the fresh culture of these clones were used for the preparation of glycerol stocks. These clones were used as a source of enzymatic polypeptide of Seq ID nos. 1, 3, 5 and 7 for subsequent biocatalysis studies.

### Example 2

### Cloning and Expression Analysis of Oxidoreductase Enzymes Derived from Genomic DNA

DNA sequences deduced from the polypeptide sequence as shown in sequence id nos. 6, 8, 9, 10 and 13 as per table no. 1 were PCR amplified with the respective primers as per Table no. 1B from S. cerevisiae and those of sequence Id nos. 11 & 12 were PCR amplified with the respective primers from E. coli for expression in E. coli. These amplified PCR products were purified and subjected to restriction digestion with the internally digesting enzyme to check the PCR product. Correct band sized PCR products corresponding to Sequence Id No. 9, 11, 12 and 13 were subjected to restriction digestion with the cloning enzymes NdeI-BamHI to be ligated with NdeI-BamHI digested vector pET27b and correct band sized PCR products corresponding to Sequence Id No. 6, 8 and 10 were to be ligated with pET27b NdeI-digested blunt vector. Each of the ligated DNA were further transformed into competent E. coli cells and the transformation mixes plated on Luria agar plates containing kanamycin. The positive clones were identified on the basis of their kanamycin resistance for growth on the above Petri plates and further restriction digestion of the plasmid DNA derived from them. Clones giving desired fragment lengths of digested plasmid DNA samples were selected as putative positive clones. One each of the putative positive clones corresponding to sequence Id nos. 6, 8, 9, 10, 11, 12, 13 were selected and named as per table no. 1A. Colonies picked from these plates were grown in Luria Broth containing kanamycin and the plasmid DNA isolated from these cultures were further subjected to restriction digestion analysis using the respective restriction enzymes to confirm the correctness of each clone. Also these cultures were subjected to induction with suitable concentration (0.01-2 mM) of IPTG for expression analysis. IPTG induced cultures were lysed and clarified lysates obtained after centrifugation were subjected to SDS-PAGE analysis to confirm induced expression of polypeptide of correct size. After confirming the restriction fragment analysis and expression analysis, the fresh cultures of these clones were used for the preparation of glycerol stocks. These clones were used as a source of enzymatic polypeptide of Seq ID nos. 6, 8, 9, 10, 11, 12 and 13 as per table no. 1A for subsequent biocatalysis studies.

### Example 3

### Construction of Plasmid pZRC2G-2ZBG2.0.9c1 for Co-Expression of Oxidoreductase and Cofactor Regenerating Enzyme

A DNA sequence deduced from the polypeptide sequence as shown in Sequence Id No. 7 which was optimized for expression in E. coli and cloned in a pET27 b plasmid vector i.e. pET27bZBG13.1.1 was used for the cloning and expression of another expression cassette of DNA Sequence Id No. 6 deduced from the cloned vector pET27bZBG2.0.9 (as per table no. 1A) in a duet manner wherein both the polypeptides of sequence id nos. 6 and 7, are expressed in a single host system. The expression construct containing T7 promoter, RBS and ZBG2.0.9 gene was amplified with the Duet primers forward 1 and reverse1 using pET27bZBG2.0.9 as template. After purifying this PCR product containing T7 promoter, RBS and ZBG 2.0.9 gene was reamplified using primers forward F2 and reverse R1 containing Bpu1102 I restriction site. The obtained PCR product was then digested with the Bpu11021 and ligated in pET27bZBG13.1.1 predigested with Bpu1102I. The ligated DNA was further transformed into competent E. coli Top10F′ cells and the transformation mix was plated on Luria agar plates containing kanamycin. The positive clones were identified on the basis of their utilizing kanamycin resistance for growth on the above Petri plates and further restriction digestion analysis of the plasmid DNA derived from them. Those restriction enzymes which were supposed to digest both the vector and the gene insert obtained from such clones. One such clone which gave desired fragment lengths of digested plasmid DNA samples was selected as a positive clone and named, pZRC2G-2ZBG2.0.9c1. Plasmid DNA isolated from this clone was transformed into the E. coli expression host, BL21 (DE3), and plated on kanamycin containing Luria Agar plates followed by incubation at 37° C. for overnight. Colonies picked from this plate were grown in Luria Broth containing kanamycin, and the plasmid DNA isolated from these cultures were further subjected to restriction digestion analysis using the respective restriction enzymes to confirm the correctness of the clone. Also these cultures were subjected to induction with suitable concentration (0.01-2 mM) of IPTG. IPTG induced cultures were lysed and clarified lysates obtained after centrifugation were subjected to SDS-PAGE analysis to confirm induced expression of polypeptide of correct size. After confirming the restriction fragment analysis and expression analysis, the fresh culture of this clone known as, pZRC2G-2ZBG2.0.9c1 BL21(DE3), was used for the preparation of glycerol stocks. This clone pZRC2G-2ZBG2.0.9c1 BL21(DE3), was used as a source of enzymatic polypeptide of Seq ID no 7 and Seq ID No. 6 for subsequent biocatalysis studies.

TABLE NO. 1A

| Clone no. | Sequence Id | Genbank Ids | pET11a clones | pET27b clones |
|---|---|---|---|---|
| 1 | sequence Id no 1 | NP_579689.1 | pET11aZBG5.1.1 | pET27bZBG5.1.1 |
| 2 | sequence Id no 2 | AAW06921.1 | pET11aZBG6.4.1 | |
| 3 | sequence Id no 3 | NP_013953.1 | pET11aZBG2.0.1 | pET27bZBG2.0.1 |
| 4 | sequence Id no 4 | AAA21973.1 | pET11aZBG25.1.1 | |
| 5 | sequence Id no 5 | BAH28833.1 | pET11aZBG8.1.1 | pET27bZBG8.1.1 |
| 6 | sequence Id no 6 | NP_012630.1 | | pET27bZBG2.0.9 |
| 7 | sequence Id no 7 | AAX31145.1 | pET11aZBG13.1.1 | pET27bZBG13.1.1 |
| 8 | sequence Id no 8 | NP_014068 | | pET27bZBG2.0.8 |
| 9 | sequence Id no 9 | NP_011330 | | pET27bZBG2.0.11 |
| 10 | sequence Id no 10 | NP_011972.1 | | pET27bZBG2.0.5 |
| 11 | sequence Id no 11 | ACB04098.1 | | pET27bZBG1.1.22 |
| 12 | sequence Id no 12 | ACB01380.1 | | pET27bZBG1.1.2 |
| 13 | sequence Id no 13 | NP_014763.1 | | pET27bZBG2.0.4 |

TABLE NO 1B

| | Sequence | |
|---|---|---|
| Sr. No. | Id | Primer sequence |
| 1 | Sequence | Forward1: 5′GGTTCCTAAGTTTTACAAAC3′ |
| | Id No. 6 | Reverse1: 5′TTATGGCGCGTCTGTGCATTC3′ |
| 2 | Sequence | Forward1: 5′GACTGACTTGTTTAAACCTCT3′ |
| | Id No. 8 | Reverse1: 5′CTAATTGTCAAAAGCTATCCTGGC3′ |
| 3 | Sequence | Forward1: 5′CGCCATATGTGCGATTCTCCTGCAACGAC3′ |
| | Id No. 9 | Reverse1: 5′CGCGGATCC TCAAACTACACGGGAGAAATGC3′ |
| 4 | Sequence | Forward1: 5′GTCTTCACTGGTTACTCTTAAT3′ |
| | Id No. 10 | Reverse1: 5′AGTGGGGAATTTACCATCCAACC3′ |
| 5 | Sequence | Forward1: 5′GAATTCCATATGGCTAATCCAACCGTTATTAAG3′ |
| | Id No. 11 | Revrese1: 5′CGCGGATCCTTAGCCGCCGAACTGGTCAGGATC3′ |
| 6 | Sequence | Forward1: 5′CGCCATATGGCTATCCCTGCATTTGGTTTAG3′ |
| | Id No. 12 | Reverse1: 5′CGCGGAACCTTAATCCCATTCAGGAGCCAGAC3′ |
| 7 | Sequence | Forward1: 5′CGCCATATGCCTGCTACTTTACATGATTC3′ |
| | Id No. 13 | Reverse1: 5′CGCGGATCCTTACTTGAATACTTCGAAAGGAG3′ |
| 8 | Duet | Forward1: 5′ATCGTATTGTACACGGCCGCATAATCGAAATTAATACGACTCACTATA3′ |
| | primers | Forward2: 5′ACCGCTGAGCTCGAACAGAAAGTAATCGTATTGTACACGGCCGCATAATCG3′ |
| | | Reverse1: 5′ATGCTAGTTATTGCTCAGCGGTGGCAGC3′ |

### Example 4

### Preparation of Enzyme at Shake Flask Condition

The recombinant/transformed E. coli clones as obtained in examples 1, 2 and 3 were cultured in 50 ml Luria Bertani (LB) medium, containing 10 g peptone, 5 g yeast extract, 10 g NaCl, per liter of water along with, 75 μg/ml kanamycin for clones 1, 3, 7, 8, 9, 10, 11, 12 and 13, or 100 μg/ml ampicillin for clones 2, 4 and 5 and cultivated for at least 16 h at 37° C. with shaking at 200 rpm. These cultures were used for inoculation into 750 ml LB medium containing 75 μg/ml kanamycin for clones 1, 3, 7, 8, 9, 10, 11, 12 and 13, or 100 μg/ml ampicillin for clones 2, 4, 5. Expression of protein was induced with 2 mM Iso-propyl β-D-thiogalactopyranoside (IPTG), when culture OD₆₀₀ reached 0.6 to 0.8 and the cultures were continued to being shaken at 200 rpm, at 37° C. for at least 16 h. Cells were harvested by centrifugation for 15 min at 7000 rpm at 4° C. and supernatant discarded. The cell pellet was re-suspended in cold 100 mM Potassium Phosphate Buffer (pH 7.0) (KPB) and harvested as mentioned above. Washed cells were re-suspended in 10 volumes of cold 100 mM KPB (pH 7.0) containing 1 mg/ml lysozyme, 1 mM PMSF and 1 mM EDTA and homogenous suspension subjected to cell lysis by ultrasonic processor (Sonics), white maintained temperature at 4° C. Cell debris was removed by centrifugation for 60 min at 12000 rpm at 4° C. The clear crude lysate supernatant (cell free extract) was lyophilized (VirTis, under Vacuum—80 to 25 m torr at temperature −80° C. to −60 C for 48-72 h) and the crude lyophilized powder stored at below 4° C. for further enzymatic reaction.

### Example 5

### Screening for oxidorectudases for reducing 4-oxo-4-[3-(trifluoromethyl)-5,6-dihydro[1,2,4]triazolo[4,3-a]pyrazin-7(8H)-yl]-1-(2,4,5-trifluorophenyl)butan-2-one

Different oxidoreductase genes of examples 1 and 2 that were over-expressed in E. coli were used in enzymatic screening for reducing oxo-4-[3-(trifluoromethyl)-5,6-dihydro[1,2,4]triazolo[4,3-a]pyrazin-7(8H)-yl]-1-(2,4,5-tri fluorophenyl)butan-2-one prepared as per WO2010/032264. For screening, the crude lyophilized powder of oxidoreductases which was previously obtained from about 240 mg induced cells was used to charge the reaction containing 100 mM Potassium phosphate buffer (pH 7.0), 7.6 mM β Nicotinamide adenine dinucleotide phosphate disodium salt (NADP⁺) or 9 mM of β Nicotinamide adenine dinucleotide free acid (NAD⁺), 100 μl isopropyl alcohol containing 10 mg (0.0246 mmoles) of 4-oxo-4-[3-(trifluoromethyl)-5,6-dihydro[1,2,4]triazolo[4,3-a]pyrazin-7(8H)-yl]-1-(2,4,5-tri fluorophenyl)butan-2-one. The homogenous screening reaction mixture was incubated for 24-48 h at 37° C.±0.5° C. under shaking condition, 200 rpm. At the end of reaction, the reaction mixture was extracted with equal volume of ethyl acetate. The separated organic phase thus obtained was analyzed on thin layer chromatography with reference to corresponding chemically synthesized racemic alcohol 3-hydroxy-1-[3-(trifluoromethyl)-5,6-dihydro[1,2,4]triazolo[4,3-a]pyrazin-7(8H)-yl]-1-(2,4,5-trifluorophenyl)butan-1-one. The purity was further analyzed by HPLC and chiral purity was analyzed by chiral HPLC method as mentioned below for the determination of enantioselectivity of formed alcohol prepared by screened crude lyophilized enzymes

A chiral HPLC analysis was carried out on Chiralcel OJ′H (250×4.6 mm, 5μ), where 5 μl sample was loaded on the column with n-Hexane as mobile phase and eluted with 0.05% TFA in Alcohol (90:10) at 30° C. temperature. The column was run for 50 mins at 0.8 mL/min flow rate. Two peaks of enantiomers appeared at retention times for peak 1 (P1) of about 31.0 min and second peak (P2) of about 35.0 min upon analysis of the chemically synthesized racemic alcohol 3-hydroxy-1-[3-(trifluoromethyl)-5,6-dihydro[1,2,4]triazolo[4,3-a]pyrazin-7(8H)-yl]trifluorophenyl)butan-1-one. Same methodology was adopted for the determination of the chiral configuration of enzymatically prepared alcohol product. Results are described in table 2.

TABLE 2

| | |
|---|---|
| % area on | |
| chiral HPLC | ee (%) of single |

| Sequence ID | P1 | P2 | enantiomer |
|---|---|---|---|
| Sequence Id no 1 | 99.84 | 0.16 | 99.68 |
| Sequence Id no 2 | 78.52 | 21.48 | 57.04 |
| Sequence Id no 3 | 97.63 | 2.37 | 95.26 |
| Sequence Id no 4 | 2.18 | 97.82 | 95.64 |
| Sequence Id no 5 | 16.71 | 83.29 | 66.58 |
| Sequence Id no 6 | 99.18 | 0.82 | 98.36 |
| Sequence Id no 8 | 43.18 | 56.82 | 13.64 |
| Sequence Id no 9 | 34.48 | 65.52 | 31.04 |
| Sequence Id no10 | 17.69 | 82.31 | 64.62 |
| Sequence Id no 11 | 4.51 | 95.49 | 90.98 |
| Sequence Id no 12 | 35.35 | 64.65 | 29.3 |
| Sequence Id no 13 | 20.59 | 79.41 | 58.82 |

### Example 6

### Preparation of Enzyme at Fermentor Level

### pET27bZBG2.0.9

Fermentation was carried out in agitated and aerated 30 L fermentor with 10 L of growth medium containing; Glucose 10 g/L, Citric acid 1.7 g/L, Yeast extract 10 g/L, Potassium di-hydrogen phosphate 13.3 g/L, Di-ammonium hydrogen phosphate 4 g/L, Magnesium sulfate heptahydrate 1.2 g/L, Trace metal solution 20 ml/L (comprised: 0.162 g/L Ferrous chloride hexahydrate, 0.0094 g/L Zinc chloride, 0.12 g/L, Cobaltous chloride, 0.012 g/L sodium molybdate dihydrate, 0.006 g/L Calcium chloride dihydrate, 2.40 g/L cupric chloride dihydrate, 0.5 g/L Boric acid) and kanamycin monosulfate 75 mg/L. The recombinant E. coli with pET27bZBG2.0.9 with late exponential cultures was used to inoculate fermentor to set an OD₆₀₀ of 0.5. The aeration was maintained at 50-70% saturation with 5-15 L/min of dissolved oxygen and agitated at 200-1000 rpm. The pH of the culture was maintained at 6.8±0.2 with 12.5% (v/v) ammonium hydroxide solution. Growth of the culture was maintained with a feed solution of growth medium containing; Glucose 700 g/L, Yeast extract 50 g/L, Trace metal 20 ml/L, Magnesium sulfate heptahydrate 10 g/L. Expression of protein was induced with Iso-propyl β-D-thiogalactopyranoside (IPTG) at the final concentration of 0.1 mM/g of DCW (Dry cell weight), when culture OD₆₀₀ reaches around 50.0±2.0. The fermentation continued further for another 12±2 hrs with feed solution of production medium containing Glucose 200 g/L, Yeast extract 200 g/L and kanamycin monosulfate 750 mg/L. The culture was slowly chilled to 10-15° C. and broth harvested by centrifugation 6500 rpm for 30 min at 4° C. Cell pellet collected after washing with 0.05M potassium phosphate buffer (pH 7.0) by centrifugation at 8000 rpm for 30 min at 4° C. Cells were stored at 4° C. or preserved at −70° C. with suitable cryoprotectant, such as 20% glycerol in 50 mM KPB buffer (pH 7.0), until used for the mentioned biocatalytic conversion.

For the preparation of crude lyophilized enzyme, the cell pellet was suspended in 10 volumes of pre-chilled 0.05M potassium phosphate buffer (pH 7.0). The homogenous single cell preparation was subjected to cell disruption by passing though high pressure homogenizer at 1000±100 psig at 4° C., in subsequent two cycles. The resulting homogenate was clarified by centrifugation at 8000 rpm for 120 min. The clear supernatant thus obtained was collected and subjected to lyophilization (VirTis, under Vacuum 80 to 25 m torr at temperature −80° C. to −60° C. for 48-72 h). The crude lyophilized powder thus obtained was used further for biocatalytic conversions.

### Example 7

### Preparation of Enzyme at Fermentor Level

### pZRC2G-2ZBG2.0.9C1

Fermentation was carried out in agitated and aerated 30 L fermentor with 10 L of growth medium containing; Glucose 10 g/L, Citric acid 1.7 g/L, Yeast extract 10 g/L, Di-Potassium hydrogen phosphate 4 g/L, Magnesium sulfate heptahydrate 1.2 g/L, Trace metal solution 20 ml/L (comprised: 0.162 g/L Ferrous chloride hexahydrate, 0.0094 g/L Zinc chloride, 0.12 g/L; Cobaltous chloride, 0.012 g/L sodium molybdate dihydrate, 0.006 g/L Calcium chloride dihydrate, 2.40 g/L cupric chloride dihydrate, 0.5 g/L Boric acid) and kanamycin monosulfate 75 mg/L. The recombinant E. coli with desired gene (as mentioned in example 3) with late exponential cultures was used to inoculate fermentor to set 0.5 OD₆₀₀.

The aeration was maintained at 50-70% saturation with 5-15 L/min of dissolved oxygen and agitated at 200-1000 rpm. The pH of the culture was maintained at 6.8±0.2 with 12.5% (v/v) ammonium hydroxide solution. Growth of the culture was maintained with a feed solution of growth medium containing; Glucose 700 g/L, Yeast extract 50 g/L, Trace metal 20 ml/L, Magnesium sulfate heptahydrate 10 g/L, kanamycin monosulfate 750 mg/L. Expression of protein was induced with Iso-propyl β-D-thiogalactopyranoside (IPTG) at the final concentration of 0.1 mM/g of DCW (Dry cell weight), when culture OD₆₀₀ reaches around 50.0±2.0. The fermentation continued further for another 12±2 hrs with feed solution of production medium containing Glucose 200 g/L, Yeast extract 200 g/L and kanamycin monosulfate 750 mg/L. The culture was slowly chilled to 10-15° C. and broth harvested by centrifugation 6500 rpm for 30 min at 4° C. Cell pellet collected after washing with 0.05M potassium phosphate buffer (pH 7.0) by centrifugation at 8000 rpm for 30 min at 4° C. Cells were stored at 4° C. or preserved at −70° C. with suitable cryoprotectant, such as 20% glycerol in 50 mM KPB buffer (pH 7.0), until used for the mentioned biocatalytic conversion.

For the preparation of crude lyophilized enzyme, the cell pellet was suspended in 10 volumes of pre-chilled 0.05M potassium phosphate buffer (pH 7.0). The homogenous single cell preparation was subjected to cell disruption by passing though high pressure homogenizer at 1000±100 psig at 4° C., in subsequent two cycles. The resulting homogenate was clarified by centrifugation at 8000 rpm for 120 min. The clear supernatant thus obtained was collected and subjected to lyophilization (VirTis, under Vacuum—80 to 25 m torr at temperature −80° C. to −60 C for 48-72 h). The crude lyophilized powder thus obtained was used further for biocatalytic conversions.

### Example 8

### Enzyme Activity of Oxidoreductase and Glucose Dehydrogenase

The oxidoreductase activity of clear crude lysate pET27bZBG2.0.9 and pZRC2G-2ZBG2.0.9C1 obtained in example 2 and 3 was assayed spectophotometrically in an NAD(P)H dependent assay at 340 nm at 25° C. One ml standard assay mixture comprised of 100 mM KPB (pH 7.0), 0.1 mM NAD(P)H, and 2.5 mM 4-oxo-4-[3-(trifluoromethyl)-5,6-dihydro[1,2,4]triazolo[4,3-a]pyrazin-7(8H)-yl]-1-(2,4,5-trifluorophenyl)butan-2-one. The reaction was initiated by addition of 100 μl of crude lysate of oxidoreductase and monitored up to 10 min. One Unit (U) of enzyme was defined as the amount of enzyme required to generate 1 mole of NAD(P)H in 1 min. The enzyme activity of cell free extract of pET27bZBG2.0.9 was determined to be 0.15 U/ml and that of cell free extract of pZRC2G-2ZBG2.0.9C1 to be 0.09 U/ml.

The glucose dehydrogenase (GDH) activity of clear crude lysate obtained in example 1 was assayed spectophotometrically in an NAD(P)H depended assay at 340 nm at 25° C. The 1.0 ml standard assay mixture comprised of 100 mM KPB (pH 7.8), 2 mM NAD(P) and 0.1M Glucose. The reaction was initiated by addition of 100 μl with suitable dilution of crude lysate and monitored up to 10 min. One unit (U) of enzyme was defined as the amount of enzyme required to oxidized 1 μmole of NAD(P)H in 1 min. The glucose dehydrogenase activity of cell free extract of pET27bZBG13.1.1 was determined to be 47 U/ml and of pZRC2G-2ZBG2.0.9C1 was determined to be 45.0 U/ml.

### Example 9

### Synthesis of (S)-3-hydroxy-1-[3-(trifluoromethyl)-5,6-dihydro[1,2,4]triazolo[4,3-a]pyrazin-7(8H)-yl]-1-(2,4,5-trifluorophenyl)butan-1-one from sodium salt of 4-oxo-4-[3-(trifluoromethyl)-5,6-dihydro[1,2,4]triazolo[4,3-a]pyrazin-7(8H)-yl]-1-(2,4,5-trifluorophenyl)butan-2-one using oxidoreductase in an enzyme coupled cofactor regeneration system

Into a 250 ml round bottom flask equipped with a thermometer inlet a pH probe and an overhead stirrer, Glucose 6.28 gm (0.0349 moles) and β-Nicotinamide adenine dinucleotide phosphate disodium salt (10 mg) was dissolved in 100 ml of water. Glucose Dehydrogenase lyophilized powder from example 4 (pET27bZBG13.1.1, 12.5 gm) was added to the reaction mixture to get suspension. 50 gm cells prepared as mentioned in the above example no 6 (pET27BZBG2.0.9) suspended in 50 ml water was added to the reaction mixture and homogeneous preparation was incubated at 25-30° C. under stirring condition. 10 gm (0.02331 moles) of substrate, i.e., sodium salt of 4-oxo-4-[3-(trifluoromethyl)-5,6-dihydro[1,2,4]triazolo[4,3-a]pyrazin-7(8H)-yl]-1-(2,4,5-trifluorophenyl)butan-2-one prepared as per WO2010/032264 was added in portions. Since this is a pH driven reaction (where pH is being maintained in the range of 7.0 to 8.0) and the substrate is basic in nature, substrate addition to the reaction mixture is to carried out in a regulated, step-by-step manner in presence of NaOH, over a period of 3-4 hours, making the total volume of the reaction mixture to 200 ml. The progress of the reaction was observed on TLC. During 25 to 30 hrs, gradually the substrate almost disappeared and the product spot was seen. Reaction mixture was extracted twice in equal volumes of ethyl acetate and upon evaporating the solvent the desired product was obtained in 60% yield.

The product was further analyzed by HPLC analysis showing an HPLC purity of >90% of the corresponding alcohol, followed by chiral HPLC analysis (as described in example no 5) showing an enantiomeric excess of >99% of single enantiomer.

The chiral configuration of this enzymatically synthesized alcohol, which appears as P1 in chiral HPLC analyses, is found to be (S), based on the discussion given in the example no. 19 below.

### Example 10

### Synthesis of (S)-3-hydroxy-1-[3-(trifluoromethyl)-5,6-dihydro[1,2,4]triazolo[4,3-a]pyrazin-7(8H)-yl]-1-(2,4,5-trifluorophenyl)butan-1-one from sodium salt of 4-oxo-4-[3-(trifluoromethyl)-5,6-dihydro[1,2,4]triazolo[4,3-a]pyrazin-7(8H)-yl]-1-(2,4,5-trifluorophenyl)butan-2-one using crude lysate of whole-cell catalyst co-expressing oxidoreductase and glucose dehydrogenase

Into a 1000 ml round bottomed flask equipped with a thermometer, an inlet, a pH probe and an overhead stirrer, Glucose (6.28 gm, 0.0349 moles) and 13-Nicotinamide adenine dinucleotide phosphate disodium salt (10 mg) was dissolved in 50 ml of water. 50 gm cells prepared as mentioned in the above example no 7 suspended in 500 ml water was subjected to cell lysis and clear cell free extract was added in the reaction mixture. The homogeneous reaction preparation was incubated at 25-30° C. under stirring condition. 10 gm (0.02331 moles) of Sodium salt of 4-oxo-4-[3-(trifluoromethyl)-5,6-dihydro[1,2,4]triazolo[4,3-a]pyrazin-7(8H)-yl]-1-(2,4,5-trifluorophenyl)butan-2-one prepared as per WO2010/032264 was added in portions to the reaction mixture by maintaining pH of the reaction at 7.0 to 8.0 as has already been explained in example 9 above. The progress of the reaction was observed on TLC. During 25 to 30 hrs the starting material was almost disappeared and product spot was seen. Reaction mixture was extracted twice in equal volumes of Ethyl acetate and upon evaporating the solvent the desired product was obtained in 72% yield.

The product was future analyzed by HPLC analysis followed by chiral HPLC analysis (as described in example no 5). Which showed >90% HPLC purity of corresponding alcohol and >99% ee of single Enantiomer.

The chiral configuration of this enzymatically synthesized alcohol, which appears as P1 in chiral HPLC analyses, is found to be (S), based on the discussion given in the example no. 19 below.

### Example 11

### Synthesis of (S)-3-hydroxy-1-[3-(trifluoromethyl)-5,6-dihydro[1,2,4]triazolo[4,3-a]pyrazin-7(8H)-yl]-1-(2,4,5-trifluorophenyl)butan-1-one from sodium salt of 4-oxo-4-[3-(trifluoromethyl)-5,6-dihydro[1,2,4]triazolo[4,3-a]pyrazin-7(8H)-yl]-1-(2,4,5-trifluorophenyl)butan-2-one using Whole-Cell Catalyst co-expressing oxidoreductase and glucose dehydrogenase at a large scale

Into a 1000 ml round bottomed flask equipped with thermometer inlet, pH probe and overhead stirrer Glucose (15.66 gm, 0.087 moles) and 13-Nicotinamide adenine dinucleotide phosphate disodium salt (12.5 mg) was dissolved in 100 ml of water. 250 gm whole cells prepared as mentioned in above example no 7 suspended in 250 ml water was added to the reaction mixture followed by 12.5 ml Toluene. The homogeneous reaction preparation was incubated at 25-30° C. under stirring condition. 25 gm (0.5827 moles) of Sodium salt of 4-oxo-4-[3-(trifluoromethyl)-5,6-dihydro[1,2,4]triazolo[4,3-a]pyrazin-7(8H)-yl]-1-(2,4,5-trifluorophenyl)butan-2-one prepared as, per WO2010/032264 was added in portions to the reaction mixture by maintaining pH of the reaction at 7.0 to 8.0 as has already been explained in example 9 above. The progress of the reaction was observed on TLC. During 25 to 30 hrs the starting material was almost disappeared and product spot was seen. Reaction mixture was extracted twice in equal volumes of ethyl acetate and upon evaporating the solvent the desired product was obtained in 72% yield.

The enzymatically prepared alcohol product was analyzed by various classical tools i.e. Melting Point (m.p.), Specific Optical Rotation (SOR), Infra Red Spectroscopy (IR) and Nuclear Magnetic Resonance spectroscopy (NMR) and ESI-MS with the following results—

m.p.; 116-120° C.

SOR [α]_{D}²⁵: 23.2° (c=1, CHCl₃)

IR (cm⁻¹): 3468, 1626, 1519

ESI-MS: 409 (M+H)⁺

¹H NMR (400 MHz, DMSO-D₆): δ 2.45-2.49 (m, 1H), 2.65-2.78 (m, 3H), 3.89-3.99 (m, 2H), 4.01-4.09 (m, 2H), 4.21-4.22 (m, 1H), 4.86-5.05 (overlapping m, 3H), 7.38-7.47 (m, 2H).

¹³C NMR (100 MHz, DMSO-D₆): δ 35.4, 37.4, 38.3, 40.1, 41.4, 42.2, 43.0, 43.7, 67.3, 105.4, 114.5, 117.1, 119.5, 123.0, 142.3, 144.4, 146.5, 148.8, 151.0, 154.6, 156.9, 170.2.

The product was further analyzed by HPLC and chiral HPLC analysis (as described in example 5), which showed 96.1% HPLC purity of corresponding alcohol and 99.7% chiral purity of single enantiomer.

The chiral configuration of this enzymatically synthesized alcohol, which appears as P1 in chiral HPLC analyses, is found to be (S), based on the discussion given in the example no. 19 below.

### Example 12

### Chemical preparation of (S)-3-(methanesulfonyloxy)-1-(3-(trifluoromethyl)-5,6-dihydro-[1,2,4]triazolo[4,3-a]pyrazin-7(8H)-yl)-4-(2,4,5-trifluorophenyl)butan-1-one

In a dry, 25 mL round bottom flask (S)-3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one (0.25 g) and dichloromethane were charged at 25-30° C. and the reaction mixture was cooled at 0-5° C. Subsequently, N,N-diisopropylethyl amine (DIPEA, 0.21 mL) was added slowly at 0-5° C. into the reaction mixture. After that methanesulfonyl chloride (0.076 mL) dissolved in dichloromethane was added slowly at 0-5° C. and reaction mixture was stirred for 1.5 h at 0-5° C. Then again methanesulfonyl chloride (0.038 mL) dissolved in dichloromethane was added slowly at 0-5° C. and the reaction mixture was stirred for 1.0 h at 0-5° C. Reaction mixture was diluted with dichloromethane and it was transferred into a separating funnel. The reaction mixture was washed with dil. aqueous HCl solution, saturated sodium bicarbonate solution, water and brine. The organic layer was collected and dried over anhydrous sodium sulfate. Solvent was distilled out at reduced pressure to obtain the title compound (Wt.—0.298 g, % Yield—100%, % Purity by HPLC—91.5%).

### Example 13

### Chemical preparation of (S)-3-(methanesulfonyloxy)-1-(3-(trifluoromethyl)-5,6-dihydro-[1,2,4]triazolo[4,3-a]pyrazin-7(8H)-yl)-4-(2,4,5-trifluorophenyl)butan-1-one

In a dry, 100 mL round bottom flask (S)-3-hydroxy-1-(3-(trifluoromethyl)-5,6-dihydro-[1,2,4]triazolo[4,3-a]pyrazin-7(8H)-yl)-4-(2,4,5-trifluorophenyl)butan-1-one (4.0 g) and dichloromethane were charged at 25-30° C. and the reaction mixture was cooled at 0-5° C. Subsequently, N,N-diisopropylethyl amine (DIPEA, 3.3 mL) was added slowly at 0-5° C. into the reaction mixture. After that methanesulfonyl chloride (1.2 mL) dissolved in dichloromethane was added slowly at 0-5° C. and reaction mixture was stirred for 1.5 h at 0-5° C. Then again N,N-diisopropylethyl amine (DIPEA, 1.7 mL) and methanesulfonyl chloride (0.6 mL) dissolved in dichloromethane were added at 0-5. The reaction mixture was stirred for 1.0 h at 0-5° C. It was diluted with dichloromethane and it was transferred into a separating funnel. The reaction mixture was washed with dil. aqueous HCl solution, saturated sodium bicarbonate solution, water and brine. The organic layer was collected and dried over anhydrous sodium sulfate. Solvent was distilled out at reduced pressure to obtain the title compound (Wt.—4.7 g, % Yield—98.5, % Purity by HPLC—95.8%, Chiral Purity by HPLC—>99.5%).

¹H NMR (400 MHz, DMSO-D₆): [symbol] 2.82-3.13 (m, 7H), 3.95-3.96 (m, 2H), 4.06-4.15 (m, 1H), 4.19-4.24 (m, 1H), 4.88-4.93 (m, 1H), 4.98-5.03 (m, 1H), 5.16-5.21 (m, 1H), 7.44-7.55 (m, 2H).

IR (cm⁻¹): 3043, 1658, 1525 ESI-MS: 487 (M+H)⁺

SOR [α]_{D}²⁵: 11.5° (c=1, CHCl₃)

### Example 14

### Chemical preparation of (R)-3-azido-1-[3-(trifluoromethyl)-5,6-dihydro[1,2,4]triazolo[4,3-a]pyrazin-7(8H)-yl]-4-(2,4,5-trifluorophenyl)butan-1-one

In a 25 mL round bottom flask (S)-3-(methanesulfonyloxy)-1-(3-(trifluoromethyl)-5,6-dihydro-[1,2,4]triazolo[4,3-a]pyrazin-7(8H)-yl)-4-(2,4,5-trifluorophenyl)butan-1-one (0.280 g) and dimethylformamide (1 mL) were charged. Subsequently, into the reaction mixture sodium azide (93 mg) was added at 25-30° C. and reaction mixture was stirred for 2 h and heated to 40-42° C. After 3 h, sodium azide (37 mg) was added and the reaction mixture was further stirred for 3 h at 40-42° C. Subsequently, the reaction mixture was cooled to 25-30° C. To the reaction mixture again sodium, azide (37 mg) was added and stirred for 14 h at 25-30° C. Reaction mixture was dumped into cold water. It was extracted with ethyl acetate. The organic layer was washed with water and brine solution. The organic layer was dried over anhydrous sodium sulfate. It was distilled out at reduced pressure to obtain the title compound (Wt.—0.228 g, % yield—91.6, % Purity by HPLC—21.4%).

### Example 15

### Chemical preparation of (R)-3-azido-1-[3-(trifluoromethyl)-5,6-dihydro[1,2,4]triazolo[4,3-a]pyrazin-7(8H)-yl]-4-(2,4,5-triflorophenyl)butan-1-one

In a 100 mL round bottom flask (S)-3-(methanesulfonyloxy)-1-(3-(trifluoromethyl)-5,6-dihydro-[1,2,4]triazolo[4,3-a]pyrazin-7(8H)-yl)-4-(2,4,5-trifluorophenyl)butan-1-one (4.0 g) and dimethylformamide (10 mL) were charged. Subsequently, into the reaction mixture sodium azide (1.32 g) was added at 25-30° C. and reaction mixture was stirred for 2 h and heated to 40-42° C. After 3 h, sodium azide (0.530 g) was added and the reaction mixture was further stirred for 3 h at 40-42° C. Subsequently, the reaction mixture was cooled to 25-30° C. To the reaction mixture again sodium azide (0.530 g) was added and stirred for 14 h at 25-30° C. Reaction mixture was dumped into cold water. It was extracted with ethyl acetate. The organic layer was washed with water and brine solution. The organic layer was dried over anhydrous sodium sulfate. It was distilled out at reduced pressure to obtain the title compound (Wt.—3.2 g, % yield—91.6%).

### Example 16

### Chemical preparation of pure (R)-3-azido-1-[3-(trifluoromethyl)-5,6-dihydro[1,2,4]-triazolo[4,3-a]pyrazin-7(8H)-yl]-4-(2,4,5-trifluorophenyl)butan-1-one

Crude (R)-3-azido-1-[3-(trifluoromethyl)-5,6-dihydro[1,2,4]-triazolo[4,3-a]pyrazin-7(8H)-yl]-4-(2,4,5-trifluorophenyl)butan-1-one (3.1 g) was purified by column chromatography over silica gel (100-200 mesh) using DIPE:EA (4:6) as an eluent (Wt. 0.565 g, % Purity by HPLC—83.0%).

¹H NMR (400 MHz, CDCl₃): [symbol] 2.61-2.70 (m, 2H), 2.82-2.94 (m, 2H), 3.98-4.26 to (overlapping m, 5H), 4.95-5.10 (overlapping m, 2H), 6.93-6.97 (m, 1H), 7.10-7.16 (m, 1H).

IR (cm⁻¹): 2121, 1664, 1521 ESI-MS: 434 (M+H)⁺

SOR [α]_{D}²⁵: (−) 3.3° (c=1, CHCl₃)

### Example 17

### Chemical preparation of (R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine [Formula II]

In a 25 mL round bottom flask (R)-3-azido-1-[3-(trifluoromethyl)-5,6-dihydro[1,2,4]triazolo[4,3-a]pyrazin-7(8H)-yl]-4-(2,4,5-trifluorophenyl)butan-1-one (0.210 g), methanol and 5% Pd/C (42 mg) were taken. The reaction mixture was cooled to 0 to 5° C. and subsequently NaBH₄ (55 mg) was added. The reaction mixture was warmed to 25-30° C. and stirred for 4-6 h at 25 to 30° C. To the reaction mixture water and hyflosupercell were added. It was filtered and washed with methanol. Filtrate was taken in a 50 mL one neck round bottom flask. Solvent was distilled out at reduced pressure. Residue was dissolved in ethyl acetate and it was washed with water and brine solution. The organic layer was collected and dried over anhydrous sodium sulfate. Distilled out the solvent at reduced pressure to obtain crude (R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine (Wt.—140 mg, HPLC Purity—30.7. %). After usual chromatographic purification pure product was obtained (Wt.—6 mg, % Chiral Purity—92%).

### Example 18

### Chemical preparation of (R)-4-oxo-4-[3-(trifluoromethyl)-5,6-dihydro-[1,2,4]triazolo[4,3-a]pyrazin-7(8H)-yl]-1-(2,4,5-trifluorophenyl)butan-2-amine [Formula II]

In a 25 mL round bottom flask crude (R)-3-azido-1-[3-(trifluoromethyl)-5,6-dihydro[1,2,4]triazolo[4,3-a]pyrazin-7(8H)-yl]-4-(2,4,5-trifluorophenyl)butan-1-one (1.35 g), methanol and 5% Pd/C (270 mg) were taken. The reaction mixture was cooled to 0 to 5° C. and NaBH₄ (355 mg) was added. It was warmed to 25-30° C. and stirred for 42 h at 25 to 30° C. After that water, methanol and hyflosupercell were added into the reaction mixture and stirred for 5-10 minutes. It was filtered and washed with methanol. Filtrate was taken in a 100 mL one neck round bottom flask. Solvent was distilled out at reduced pressure and to obtain crude (R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine. After usual acid-base purification pure product was obtained (Wt.—0.852 g, % Purity by HPLC—92.8%, Chiral Purity by HPLC—>99.5%).

¹H NMR (400 MHz, CDCl₃): δ 2.58-2.80 (m, 2H), 2.82-2.95 (m, 2H), 3.64-3.69 (m, 1H),

3.70-3.98 (m, 1H), 4.07-4.22 (m, 3H), 4.88-5.06 (m, 2H), 6.88-6.94 (m, 1H), 7.10-7.16 (m, 1H).

IR (cm⁻¹): 1649, 1518

ESI-MS: 408 (M+H)⁺

### Example 19

### Determination of Chiral Configuration of the Key Compounds

The chiral configuration of the Amine compound (Examples 17 and 18) was identified through chiral HPLC analysis of racemic 4-oxo-4-[3-(trifluoromethyl)-5,6-dihydro[1,2,4]triazolo[4,3-a]pyrazin-7(8H)-yl]-1-(2,4,5-trifluorophenyl)butan-2-amine and it's (R)-isomer which is commercially known as the drug, Sitagliptin.

The RT of the product obtained in examples 17 and 18 was matching with the RT of known (R)-isomer of Sitagliptin in Chiral HPLC analysis. Therefore, it was concluded that the final amine compound obtained in above examples was (R)-isomer.

The preparation of (R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine can also be explained by following conversion steps which are based on the classical chemistry principals and are well known prior art of organic synthesis,

In Examples 17 and 18, the (R)— isomer of 4-oxo-4-[3-(trifluoromethyl)-5,6-dihydro[1,2,4]triazolo[4,3-a]pyrazin-7(8H)-yl]-1-(2,4,5-trifluorophenyl)butan-2-amine has been obtained from (R)-3-azido-1-[3-(trifluoromethyl)-5,6-dihydro[1,2,4]triazolo[4,3-a]pyrazin-7(8H)-yl]-1-(2,4,5-trifluorophenyl)butan-2-one after reduction reaction with the retention of configuration as is well known in classical chemistry. Therefore, the use of retention chemistry ensures that the compound produced in examples 14, 15 and 16 is of the (R)-configuration.

Similarly, in Examples 14, 15 and 16, the (R)-3-azido-1-[3-(trifluoromethyl)-5,6-dihydro[1,2,4]triazolo[4,3-a]pyrazin-7(8H)-yl]-1-(2,4,5-trifluorophenyl)butan-2-one has been prepared from (S)-3-(methanesulfonyloxy)-1-(3-(trifluoromethyl)-5,6-dihydro-[1,2,4]triazolo[4,3-a]pyrazin-7(8H)-yl)-4-(2,4,5-trifluorophenyl)butan-1-one by nucleophilic substitution reaction with the inversion of configuration at the chiral center i.e. from (S)-methansulfonate compound to (R)-Azido, compound as is well known in classical chemistry. Therefore, the use of inversion chemistry ensures that the compound produced in examples 12, and 13 is of the (S)-configuration.

Finally, in Examples 12 and 13, (S)-3-(methanesulfonyloxy)-1-(3-(trifluoromethyl)-5,6-dihydro-[1,2,4]triazolo[4,3-a]pyrazin-7(8H)-yl)-4-(2,4,5-trifluorophenyl)butan-1-one has been obtained from (S)-3-hydroxy-1-[3-(trifluoromethyl)-5,6-dihydro[1,2,4]triazolo[4,3-a]pyrazin-7(8H)-yl]-1-(2,4,5-trifluorophenyl)butan-1-one with the retention of configuration on the chiral center as is well known in classical chemistry. Therefore, the use of retention chemistry ensures that the compound produced in examples 9, 10 and 11 is of the (S)-configuration. This configuration has also been described in example 5 as peak 1 (P1). And therefore P1 of example 5 can be concluded to be representing the (S)-configuration of the chiral alcohol, (S)-3-hydroxy-1-[3-(trifluoromethyl)-5,6-dihydro[1,2,4]triazolo[4,3-a]pyrazin-7(8H)-yl]-1-(2,4,5 trifluorophenyl)-butan-1-one. In the same manner, peak 2 (P2) being of the opposite chirality as per the chiral analysis of racemic chiral alcohol, 3-hydroxy-1-[3-(trifluoromethyl)-5,6-dihydro[1,2,4]triazolo[4,3-a]pyrazin-7(8H)-yl]-1-(2,4,5 trifluorophenyl)-butan-1-one, discussed in example 19, can be concluded to be representing the (R)-configuration of the relevant chiral alcohol.

1. A process for the preparation of compound of formula (I) 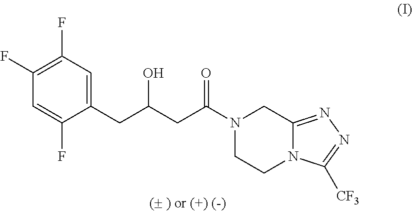 [SMILES: O=C(CC(O)Cc1cc(F)c(F)cc1F)N1CCn2c(nnc2C(F)(F)F)C1]
comprising:
a) Reacting 4-oxo-4-[3-(trifluoromethyl)-5,6-dihydro[1,2,4]triazolo[4,3-a]pyrazin-7(8H)-yl]-1-(2,4,5-trifluorophenyl)butan-2-one of formula (III): 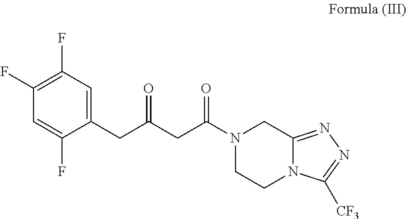 [SMILES: O=C(CC(=O)N1CCn2c(nnc2C(F)(F)F)C1)Cc1cc(F)c(F)cc1F]
with an enzyme that selectively reduces a ketone to form an alcohol, by maintaining under suitable conditions and in presence of a suitable cofactor;
b) Isolating the suitable intermediate.

2. The process as claimed in claim 1, wherein the suitable enzyme is Oxidoreductase.

3. The process as claimed in claim 1, wherein the suitable enzyme is Ketoreductase.

4. The process as claimed in claim 1, wherein the suitable enzyme is short chain dehydrogenase.

5. The process as claimed in claim 1, wherein the suitable enzyme is alcohol dehydrogenase.

6. The process as claimed in claim 1, wherein the suitable enzyme is aldoketo reductases.

7. The process as claimed in claim 1, wherein the suitable enzyme is isolated from saccharomyces, rhodotorula, pichia and E. coli.

8. The process as claimed in claim 1, wherein the suitable enzyme is isolated from species selected from saccharomyces cervisiae, rhodotorula rubra, pichia methanolica and E. coli.

9. The process as claimed in claim 1, wherein the suitable enzyme is selected from nucleotide sequence which is set forth in SEQ ID NO:1, SEQ ID NO:2, SEQ ID NO:3, SEQ ID NO:4, SEQ ID NO:5, SEQ ID NO:6, SEQ ID NO:8, SEQ ID NO:9, SEQ ID NO:10, SEQ ID NO:11, SEQ ID NO:12 and SEQ ID NO:13.

10. The process as claimed in claim 1, wherein the enzyme having nucleotide sequence is selected from nucleotide sequence which is set forth in SEQ ID NO:1, SEQ ID NO:2, SEQ ID NO:3, SEQ ID NO:4, SEQ ID NO:5, SEQ ID NO:6, SEQ ID NO:8, SEQ ID NO:9, SEQ ID NO:10, SEQ ID NO:11, SEQ ID NO:12 and SEQ ID NO:13 or its variants is cloned in a vector and subsequently expressed in a suitable recombinant whole cell.

11. The process as claimed in claim 10, wherein the recombinant whole cell further co-express polypeptide having potential to regenerate cofactor from oxidized NAD(P).

12. The process as claimed in claim 1, wherein the whole cell is selected from MTCC 5642, MTCC 5643, MTCC 5644, MTCC 5645, MTCC 5646, MTCC 5647, MTCC 5648, MTCC 5649, MTCC 5650, MTCC 5651, MTCC 5652, MTCC 5653, MTCC 5654

13. The process as claimed in claim 12, wherein the whole cell comprising an expression vector which comprises:
a) At least one region that control the replication and maintenance of said vector in the host cell;
b) first promoter operably linked to the nucleotide sequence selected from nucleotide sequences which is set forth in SEQ ID NO:1, SEQ ID NO:2, SEQ ID NO:3, SEQ ID NO:4, SEQ ID NO:5, SEQ ID NO:6, SEQ ID NO:8, SEQ ID NO:9, SEQ ID NO:10, SEQ ID NO:11, SEQ ID NO:12 and SEQ ID NO:13 or its variants encoding the oxidoreductase enzyme;
c) second promoter operably linked to the nucleotide sequence which is setforth in SEQ ID NO:7 encoding polypeptide having potential to regenerate co-factor; and
d) suitable antibiotic marker.

14. A process for the preparation of suitable intermediate of formula (I) 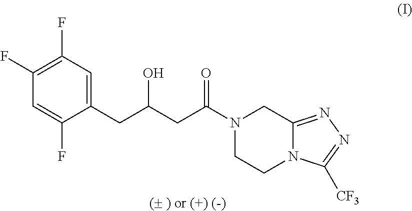 [SMILES: O=C(CC(O)Cc1cc(F)c(F)cc1F)N1CCn2c(nnc2C(F)(F)F)C1]
comprising:
a) reacting 4-oxo-4-[3-(trifluoromethyl)-5,6-dihydro[1,2,4]triazolo[4,3-a]pyrazin-7(8H)-yl]-1-(2,4,5-trifluorophenyl)butan-2-one of formula (III): 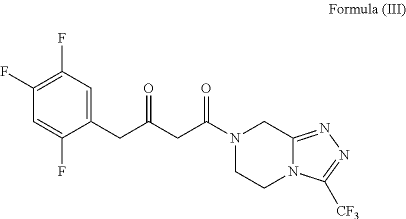 [SMILES: O=C(CC(=O)N1CCn2c(nnc2C(F)(F)F)C1)Cc1cc(F)c(F)cc1F]
with a whole cell that stereoselectively reduces a ketone to form an alcohol, by maintaining under suitable conditions and cofactor
b) isolating the suitable intermediate

15. The process as claimed in claim 14, wherein the whole cell is selected from MTCC 5642, MTCC 5643, MTCC 5644, MTCC 5645, MTCC 5646, MTCC 5647, MTCC 5648, MTCC 5649, MTCC 5650, MTCC 5651, MTCC 5652, MTCC 5653, MTCC 5654

16. The process as claimed in claim 1, wherein cofactor is continuously regenerated through enzyme based regeneration system wherein the enzyme oxidizes the suitable co-substrate to regenerate co-factor.

17. The process as claimed in claim 1, wherein the enzyme employed in co-factor regeneration is selected from glucose dehydrogenase, formate dehydrogenase, malate dehydrogenase, glucose-6-phosphate dehydrogenase, phosphite dehydrogenase.

18. The process as claimed in claim 16 wherein the enzyme employed in co-factor regeneration is glucose dehydrogenase as set forth in SEQ ID NO:7 or its variants

19. The process as claimed in claim 1, wherein cofactor is continuously regenerated through substrate based co-factor regeneration system wherein the enzyme oxidize the suitable co-substrate to regenerate co-factor.

20. The process as claimed in claim 19, wherein the enzyme is selected from oxidoreductase, ketoreductase, short chain dehydrogenase, alcohol dehydrogenase and aldoketo reductases.

21. The process as claimed in claim 19, wherein the co-substrate is isopropyl alcohol.

22. The process as claimed in claim 1, wherein the concentration of formula (III) is selected from 0.1 to 30% w/v.

23. The process as claimed in claim 1, wherein the cofactor is NAD(P)H and NAD(P).

24. The process as claimed in claim 2, wherein the pH is maintained at 5 to 9 preferably 7 to 8.

25. A vector for the expression of chiral alcohol of formula (I) which comprises
a. at least one region that control the replication;
b. suitable promoter operably linked to the desired nucleotide sequence selected from which is set forth in SEQ ID NO:1, SEQ ID NO:2, SEQ ID NO:3, SEQ ID NO:4, SEQ ID NO:5, SEQ ID NO:6, SEQ ID NO:8, SEQ ID NO:9, SEQ ID NO:10, SEQ ID NO:11, SEQ ID NO:12 and SEQ ID NO:13 or its variants; and
c. an antibiotic marker.

26. The vector as claimed in claim 25 which further comprises the polynucleotide sequence of SEQ ID NO:7 or its variants.

27. The vector as claimed in claim 25, which expresses the oxidoreductase enzyme is pET11aZBG5.1.1, pET11aZBG6.4.1, pET11aZBG2.0.1, pET11aZBG25.1.1, pET11aZBG8.1.1, pET11aZBG13.1.1, pET27bZBG5.1.1, pET27bZBG2.0.1, pET27bZBG8.1.1, pET27bZBG2.0.9, pET27bZBG13.1.1, pET27bZBG2.0.8, pET27bZBG2.0.11, pET27bZBG2.0.5, pET27bZBG1.1.22, pET27bZBG1.1.2, pET27bZBG2.0.4

28. The vector, pET27bZBG2.0.9, as claimed in claim 27 expressing the Oxidoreductase enzyme.

29. The vector, pET27bZBG13.1.1, as claimed in claim 27 expressing the Glucose dehydrogenase enzyme.

30. The vector, pZRC2G-2ZBG2.0.9C1, as claimed in claim 27 co-expressing the oxidoreductase and Glucose dehydrogenase enzymes.

31. A compound of formula

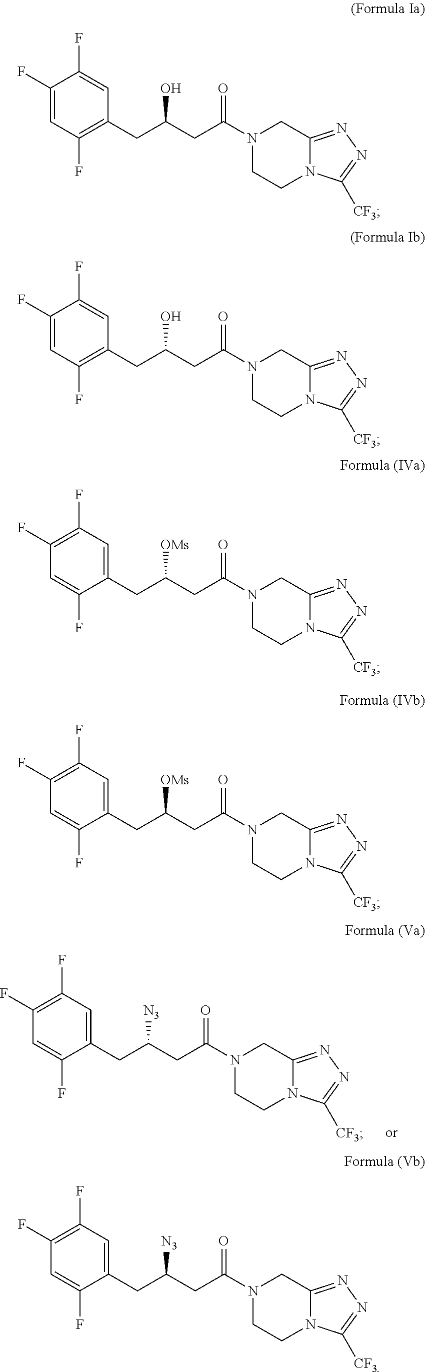

32. A process for the preparation of compound Formula (II) comprising
(a) reacting (S)-3-hydroxy-1-(3-(trifluoromethyl)-5,6-dihydro-[1,2,4]triazolo[4,3-a]pyrazin-7(8H)-yl)-4-(2,4,5-trifluorophenyl) butan-1-one of Formula (Ib) 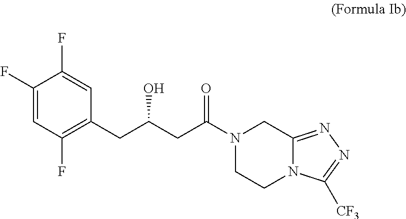 [SMILES: O=C(C[C@@H](O)Cc1cc(F)c(F)cc1F)N1CCn2c(nnc2C(F)(F)F)C1]
with methanesulfonyl chloride to obtain compound of Formula (IVa); 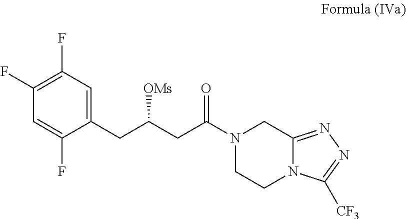 [SMILES: CS(=O)(=O)O[C@H](CC(=O)N1CCn2c(nnc2C(F)(F)F)C1)Cc1cc(F)c(F)cc1F]
(b) converting compound of Formula (IVa) to compound of Formula (Vb) by using sodium azide; 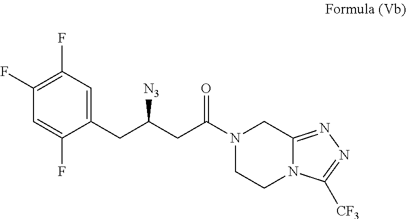 [SMILES: [N-]=[N+]=N[C@@H](CC(=O)N1CCn2c(nnc2C(F)(F)F)C1)Cc1cc(F)c(F)cc1F]
c) the compound of Formula (Vb) is converted to the compound of Formula (II) by using Pd/c and sodium borohydride.
